(19) United States
(12) Patent Application Publication
(10) Pub. No.: US 2026/0026257 A1
(43) Pub. Date: Jan. 22, 2026

(54) Functional Layer Material, Light-Emitting Device, and Display Panel

(71) Applicant: BOE TECHNOLOGY GROUP CO., LTD., Chaoyang District, Beijing (CN)

(72) Inventor: Yuqian Sun, Beijing (CN)

(21) Appl. No.: 18/998,934

(22) PCT Filed: May 31, 2024
(86) PCT No.: PCT/CN2024/096708

§ 371 Date: Jan. 27, 2025

(30) Foreign Application Priority Data
Jun. 15, 2023 (CN) 202310715885.2

(51) Int. Cl. H10K 85/60; C07D 405/12; C07D 409/12; C09K 11/02; C09K 11/06; H10K 50/11; H10K 50/15; H10K 50/18

(52) U.S. Cl. CPC H10K 85/636; C07D 405/12; C07D 409/12; C09K 11/02; C09K 11/06; H10K 85/626; H10K 85/633; C09K 2211/1007; C09K 2211/1011; C09K 2211/1014; H10K 50/11; H10K 50/15; H10K 50/181; H10K 85/615; H10K 85/622; H10K 85/6572; H10K 85/6574; H10K 85/6576

(57) ABSTRACT

A functional layer material is selected from any one of structures shown in a general formula (I). IA represents a first substitution unit including at least one fused ring aryl group; X is selected from any one of O, S and Se; Y is selected from any one of O, S, N(R₄), C(R₅R₆) and a single bond; L₁ is selected from any one of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; L₂ is selected from any one of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups.

### CROSS-REFERENCE TO RELATED APPLICATIONS

This application is the United States national phase of International Patent Application No. PCT/CN2024/096708, filed May 31, 2024, and claims priority to Chinese Patent Application No. 202310715885.2, filed Jun. 15, 2023, the disclosures of which are hereby incorporated by reference in their entireties.

### BACKGROUND OF THE INVENTION

### Field of the Invention

The present disclosure relates to the field of display technologies, and in particular, to a functional layer material, a light-emitting device and a display panel.

### Description of Related Art

Organic light-emitting diode (OLED) light-emitting devices have become the most promising new light-emitting devices in recent years due to self-luminescence, fast response speed and other advantages. During light emission of the OLED light-emitting device, holes from the anode and electrons from the cathode are emitted to a light-emitting layer included in the OLED light-emitting device, these electrons and holes are combined to form electron-hole pairs, and the formed electron-hole pairs are converted from a singlet state to a ground state to emit light.

### SUMMARY OF THE INVENTION

In an aspect, a functional layer material is provided. The functional layer material is selected from any of structures represented by a general formula (I).

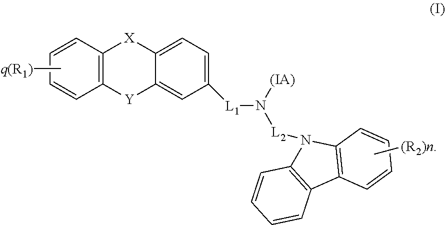

Where IA represents a first substitution unit that includes at least one fused ring aryl group; X is selected from any of O, S and Se; Y is selected from any of O, S, N(R₄), C(R₅R₆) and a single bond, and X and Y are same or different; L₁ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; L₂ is selected from any of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; and L₂ and L₁ are same or different; R₁, R₂, R₄, R₅ and R₆ are same or different, and are each independently selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups; n takes a value of a positive integer greater than or equal to 1; and q takes a value of a positive integer greater than or equal to 1.

In some embodiments, the functional layer material is selected from any of structures represented by a general formula (II).

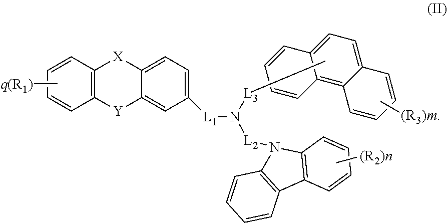

Where L₃ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; L₃ and L₁ are same or different, and L₃ and L₂ are same or different; R₃ is selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups; and m takes a value of a positive integer greater than or equal to 1.

In some other embodiments, X is oxygen and Y is a single bond; alternatively, X is sulfur and Y is a single bond.

In some other embodiments, X is oxygen and Y is oxygen; alternatively, X is oxygen and Y is sulfur.

In some other embodiments, the functional layer material is selected from any of structures represented by a general formula (III).

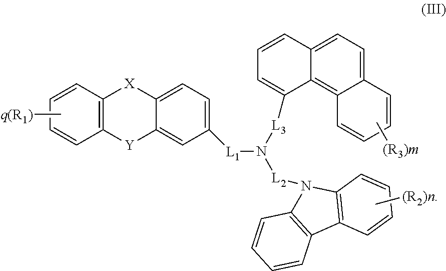

In some other embodiments, the functional layer material is selected from any of structures represented by a general formula (IV).

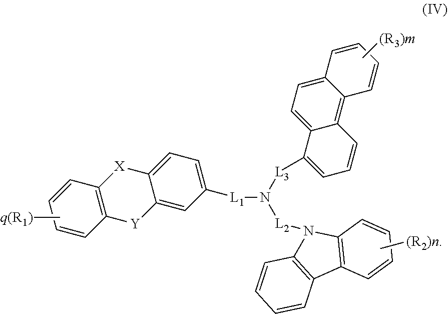

In some other embodiments, in a case where the functional layer material is selected from any of structures represented by the general formula (I), the structures represented by the general formula (I) contain at least one deuterium; alternatively, in a case where the functional layer material is selected from any of the structures represented by the general formula (II), the structures represented by the general formula (II) contain at least one deuterium; alternatively, in a case where the functional layer material is selected from any of the structures represented by the general formula (III), the structures represented by the general formula (III) contain at least one deuterium; alternatively, in a case where the functional layer material is selected from any of the structures represented by the general formula (IV), the structures represented by the general formula (IV) contain at least one deuterium.

In some other embodiments, in a case where m is greater than 2, two adjacent R₃ are bonded to be a ring.

In some other embodiments, in a case where q is greater than 2, two adjacent R₁ are bonded to be a ring.

In some other embodiments, in a case where n is greater than 2, two adjacent R₂ are bonded to be a ring.

In some other embodiments, the functional layer material is used to transport holes and/or block electrons.

In another aspect, a light-emitting device is provided and includes a cathode and an anode that are opposite, and at least one light-emitting unit disposed between the cathode and the anode. The light-emitting unit includes a light-emitting layer and a first type of functional layer disposed on a side of the light-emitting layer proximate to the anode. A material of the first type of functional layer includes the functional layer material as described in any of the above embodiments.

In some embodiments, the first type of functional layer includes a plurality of functional sub-layers, and at least one functional sub-layer in the plurality of functional sub-layers includes the functional layer material.

In some other embodiments, the plurality of functional sub-layers include an electron blocking functional layer, a hole injection functional layer and a hole transport functional layer that are stacked. The hole injection functional layer, the hole transport functional layer and the electron blocking functional layer are arranged in sequence in a direction away from the anode. In the hole injection functional layer, the hole transport functional layer and the electron blocking functional layer, at least the electron blocking functional layer includes the functional layer material.

In some other embodiments, the light-emitting layer is configured to emit blue light, and a material of the light-emitting layer includes a host material and a guest material. A structural formula of the host material is as follows:

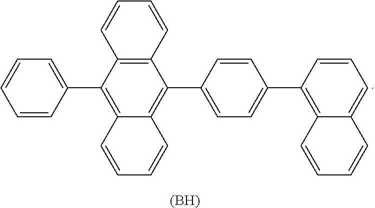

A structural formula of the guest material is as follows:

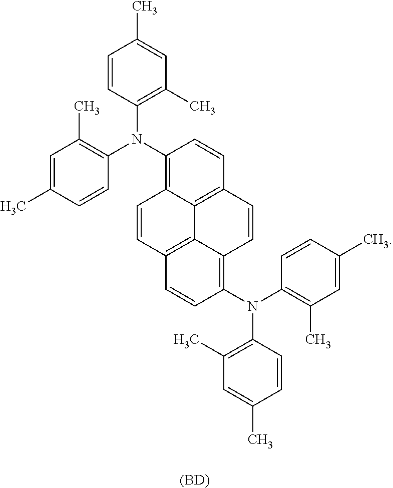 [SMILES: Cc1ccc(N(c2ccc(C)cc2C)c2ccc3ccc4c(N(c5ccc(C)cc5C)c5ccc(C)cc5C)ccc5ccc2c3c54)c(C)c1 ; "(BD)"]

In yet another aspect, a display panel is provided and includes a plurality of light-emitting devices each as described in any of the above embodiments; and further includes pixel driving circuits electrically connected to the light-emitting devices, the pixel driving circuits are used to drive the light-emitting devices to emit light.

### BRIEF DESCRIPTION OF THE DRAWINGS

In order to describe technical solutions in the present disclosure more clearly, accompanying drawings to be used in some embodiments of the present disclosure will be introduced briefly below. Obviously, the accompanying drawings to be described below are merely accompanying drawings of some embodiments of the present disclosure, and a person of ordinary skill in the art may obtain other drawings according to these drawings. In addition, the accompanying drawings to be described below may be regarded as schematic diagrams, but are not limitations on an actual size of a product, an actual process of a method and an actual timing of a signal to which the embodiments of the present disclosure relate.

FIG. 1 is a structural diagram of a light-emitting device, in accordance with some embodiments of the present disclosure;

FIG. 2 is a structural diagram of a display panel, in accordance with some embodiments of the present disclosure;

FIG. 3 is a structural diagram of another display panel, in accordance with some embodiments of the present disclosure; and

FIG. 4 is a structural diagram of a display apparatus, in accordance with some embodiments of the present disclosure.

### DESCRIPTION OF THE INVENTION

Technical solutions in some embodiments of the present disclosure will be described clearly and completely with reference to the accompanying drawings below. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as open and inclusive, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials, or characteristics described herein may be included in any one or more embodiments or examples in any suitable manner.

Hereinafter, the terms such as “first” and “second” are used for descriptive purposes only, and are not to be construed as indicating or implying the relative importance or implicitly indicating the number of indicated technical features. Thus, features defined with “first” or “second” may explicitly or implicitly include one or more of the features. In the description of the embodiments of the present disclosure, the term “a plurality of” or “the plurality of” means two or more unless otherwise specified.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

The term “about”, “substantially” or “approximately” as used herein includes a stated value and an average value within an acceptable range of deviation of a particular value. The acceptable range of deviation is determined by a person of ordinary skill in the art in consideration of the measurement in question and errors associated with the measurement of a particular quantity (i.e., limitations of the measurement system).

The term such as “parallel”, “perpendicular” or “equal” as used herein includes a stated condition and a condition similar to the stated condition. A range of the similar condition is within an acceptable range of deviation. The acceptable range of deviation is determined by a person of ordinary skill in the art in view of measurement in question and errors associated with the measurement of a particular quantity (i.e., limitations of the measurement system). For example, the term “parallel” includes absolute parallelism and approximate parallelism, and an acceptable range of deviation of the approximate parallelism may be a deviation within 5°; the term “perpendicular” includes absolute perpendicularity and approximate perpendicularity, and an acceptable range of deviation of the approximate perpendicularity may also be a deviation within 5°; and the term “equal” includes absolute equality and approximate equality, and an acceptable range of deviation of the approximate equality may be a difference between two equals being less than or equal to 5% of either of the two equals.

It will be understood that when a layer or element is referred to as being on another layer or substrate, the layer or element may be directly on the another layer or substrate, or there may be intermediate layer(s) between the layer or element and the another layer or substrate.

Exemplary embodiments are described herein with reference to sectional views and/or plane views as idealized exemplary drawings. In the accompanying drawings, thicknesses of layers and sizes of areas/regions are enlarged for clarity. Variations in shapes relative to the accompanying drawings due to, for example, manufacturing technologies and/or tolerances may be envisaged. Therefore, the exemplary embodiments should not be construed to be limited to the shapes of areas/regions shown herein, but to include deviations in the shapes due to, for example, manufacturing. For example, an etched area/region shown in a rectangular shape generally has a feature of being curved. Therefore, the areas/regions shown in the accompanying drawings are schematic in nature, and their shapes are not intended to show actual shapes of the areas/regions in a device, and are not intended to limit the scope of the exemplary embodiments.

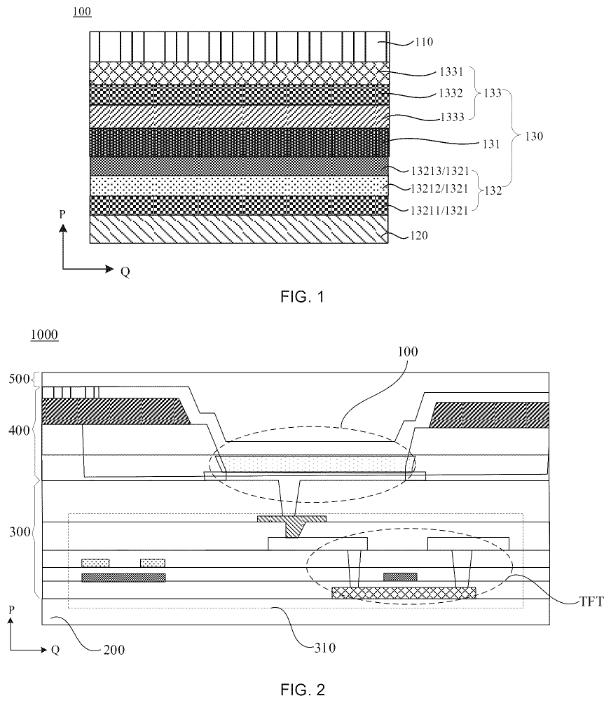

It will be noted that a symbol such as ½ appearing in the accompanying drawings of the present disclosure indicates that both a structure 1 and a structure 2 may refer to the structure shown. For example, 13211/1321 in FIG. 1 indicates that both a hole injection functional layer 13211 and a functional sub-layer 1321 may refer to the structure shown. Other similar symbols appearing in the drawings also follow the above description.

As mentioned in the background, in the organic semiconductor field, an OLED technology has attracted more and more attention from academia and industry, and has been successfully applied in commercial flat panel display and lighting industries. OLED light-emitting devices have a characteristic of self-luminescence. Accordingly, an OLED display panel and an OLED light-emitting substrate have advantages of no need for backlight source, a small thickness of a panel and light weight. Moreover, the OLED light-emitting devices also have advantages of being all solid-state, fast response speed and a wide operating temperature range.

The compounds of all organic material layers applied to the OLED light-emitting device have different compositions, which will produce great differences in overall performances of the organic light-emitting devices. With the development of the OLED light-emitting device, requirements for efficiency, power consumption, a service life and other properties of the OLED light-emitting device are becoming higher and higher, and existing organic materials can hardly meet the requirements of the OLED light-emitting device.

In light of this, some embodiments of the present disclosure provide a functional material layer F, and the functional material layer F is selected from any of structures represented by a following general formula (I).

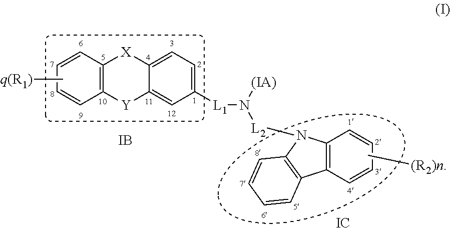

Here, IA represents a first substitution unit that includes at least one fused ring aryl group.

X is selected from any of O, S and Se.

Y is selected from any of O, S, N(R₄), C(R₅R₆) and a single bond, and X and Y are the same or different.

L₁ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups.

L₂ is selected from any of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; and L₂ and L₁ are the same or different.

R₁, R₂, R₄, R₅ and R₆ are the same or different, and are each independently selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups.

n takes a value of a positive integer greater than or equal to 1.

q takes a value of a positive integer greater than or equal to 1.

For example, a value of n is any of 1, 2, 3, 4, 5, 6, 7 and 8.

For example, a value of q is any of 1, 2, 3, 4, 5, 6 and 7.

Regarding the structure shown in the general formula (I), the following points need to be explained.

The first substitution unit represented by IA includes at least one fused ring aryl group, and the fused ring aryl group may be a naphthyl group, a phenanthryl group, and the like. That is, the number of aromatic rings fused together in the fused ring aryl group may be 2, 3, or a positive integer greater than 3.

X is selected from any of O, S and Se, where O is oxygen, S is sulfur, and Se is selenium.

Y is selected from any of O, S, N(R₄), C(R₅R₆) and a single bond, where O is oxygen, S is sulfur, N(R₄) is a group in which an atom bonded to nitrogen is substituented by R₄, C(R₅R₆) is a group in which atoms bonded to carbon are independently substituented by R₅ and R₆, and R₅ and R₆ may be the same or different.

In a case where Y is selected as a single bond, carbon 11 and carbon 12 in the IB part are directly connected by a covalent bond.

L₁ may be selected as a single bond, and in a case where L₁ is a single bond, nitrogen and carbon 1 in the IB part are directly connected by a covalent bond.

L₁ and L₂ may be selected from any of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups. Here, the Cx aryl group refers to an aryl group having x carbon (C) atoms, where x is a positive integer, and the same applies to the following. For understandings of groups such as the Cx heteroaryl group and the Cx aralkylene group, reference may be made to the above contents, and details are not repeated here. In addition, a phenyl group refers to a general name of a group left after a hydrogen atom of one carbon atom on the benzene ring is removed. A phenylene group refers to a general name of a group left after hydrogen atoms of two carbon atoms on the benzene ring are removed. For understandings of other groups such as the arylene group, the heteroarylene group and an alkylene group, reference may be made to the above contents, and details are not repeated here.

It will be noted that the aryl group may be a phenyl group. The heteroaryl group may be a furyl group, a pyranyl group, a thienyl group, a pyridinyl group, and the like. The C1 heteroaryl group refers to a group containing an aromatic ring with one carbon atom thereon. The aralkylene group refers to an alkylene group with a substituent of an aryl group. The C7 aralkylene group refers to a C1 alkylene group with a substituent of a phenyl group. The heteroaralkylene group refers to an alkylene group with a substituent of a heteroaryl group. The C2 heteroaralkylene group refers to a C1 alkylene group with a substituent of a C1 heteroaryl group.

For example, in a case where L₁ and L₂ are aralkylene groups, the connection position between nitrogen and the aralkylene group may be an alkylene group portion of the aralkylene group. Here, nitrogen is the nitrogen connected to the IB part, the IC part, and the first substitution unit IA.

For example, in a case where L₁ and L₂ are heteroaralkylene groups, the connection position between nitrogen and the heteroaralkylene group may be an alkylene group portion of the heteroaralkylene group. Here, nitrogen is the nitrogen connected to the IB part, the IC part, and the first substitution unit IA.

In a case where L₁ and L₂ are selected from substituted C6 to C30 arylene groups, substituted C1 to C30 heteroarylene groups, substituted C7 to C30 aralkylene groups, and substituted C2 to C30 heteroaralkylene groups, the types of the substituents are not limited here.

In a case where R₁, R₂, R₄, R₅ and R₆ are selected from substituted C1 to C39 alkyl groups, substituted C2 to C39 alkenyl groups, substituted C2 to C39 alkynyl groups, substituted C6 to C39 aryl groups, substituted C5 to C60 heteroaryl groups, substituted C6 to C60 aryloxy groups, substituted C1 to C39 alkoxy groups, substituted C6 to C39 arylamine groups, substituted C3 to C39 cycloalkyl groups, substituted C3 to C39 heterocyclylalkyl groups, substituted C1 to C39 alkylsilyl groups, substituted C1 to C39 alkyl boryl groups, substituted C6 to C39 aryl boryl groups, substituted C6 to C39 arylphosphino groups, and substituted C6 to C39 arylsilyl groups, the types of the substituents are not limited here.

In the general formula (I), L₁ is connected to carbon 1 in the IB part. The connection position between R₁ and the IB part shown in the general formula (I) refers to that R₁ may be connected to any of carbon 2, 3, 6, 7, 8, 9 and 12 in the IB part, that is, R₁ may be connected to any of carbon 2, 3, 6, 7, 8, 9 and 12 that has a substitution position.

In a case where the value of q is greater than or equal to 2, that is, in a case where the number of R₁ is greater than or equal to 2, the types of the q R₁ may be the same or different.

The connection position between R₂ and the IC part shown in the general formula (I) refers to that R₂ may be connected to any of carbon 1′, 2′, 3′, 4′, 5′, 6′, 7′ and 8′ in the IC part, that is, R₂ may be connected to any of carbon 1′, 2′, 3′, 4′, 5′, 6′, 7′ and 8′ that has a substitution position.

In a case where the value of n is greater than or equal to 2, that is, in a case where the number of R₂ is greater than or equal to 2, the types of the n R₂ may be the same or different.

It can be seen from the structure shown in the general formula (I) that the functional layer material F is a triarylamine-based material. By connecting the IA part, the IB part and the IC part to N, the functional layer material F is a hole-type material which may be used to transport holes and/or block electrons. For example, as shown in FIG. 1, in a structural diagram of a light-emitting device 100, the functional layer material F is used to form a first type of functional layer 132 having a hole transport function, such as an electron blocking functional layer 13213 and a hole transport functional layer 13212, and is used to transport holes to a light-emitting layer 131 (an introduction to the structure of the light-emitting device 100 may see the following contents, and details are not repeated here).

Moreover, three substitution units of the triarylamine-based material are the first substitution unit IA, a second substitution unit IB and a third substitution unit IC. Here, the heteroatom X or Y in the second substitution unit IB may improve a hole transport ability of the functional layer material F, increase the amount of holes transported to the light-emitting layer 131, improve a recombination probability of holes and electrons in the light-emitting layer 131, thereby improving an emission efficiency. Moreover, since the second substitution unit IB has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131. In this way, the luminous efficiency of the light-emitting device 100 may be effectively improved. The third substitution unit IC contains a carbazolyl group, that is,

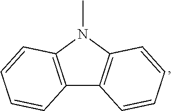

so that the functional layer material F may have a suitable highest occupied molecular orbital (HOMO) energy level, thereby reducing an energy gap (GAP) that needs to be overcome during transport of holes from the first type of functional layer 132 to the light-emitting layer 131. In this way, a voltage for driving the light-emitting device 100 to emit light may be effectively reduced, and thus the power consumption of the light-emitting device 100 may be reduced. Further, since the first substitution unit IA is a fused ring aryl group with relatively good conjugation, due to a synergistic effect of the second substitution unit IB and the third substitution unit IC, the light-emitting device 100 may have a relatively high luminous efficiency, a relatively low voltage and a long service life.

In some embodiments, the functional layer material F is selected from any of structures represented by a following general formula (II).

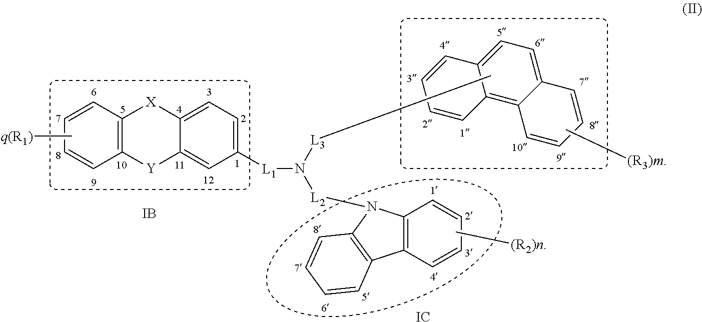

Here, L₃ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; L₃ and L₁ are the same or different, and L₃ and L₂ are the same or different.

R₃ is selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups.

m takes a value of a positive integer greater than or equal to 1.

For example, a value of m is any of 1, 2, 3, 4, 5, 6, 7, 8 and 9.

It can be understood that IA-1 in the general formula (II) is a phenanthryl group.

It will be noted that L₃ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups. In a case where L₃ is a single bond, nitrogen and one carbon atom on the phenanthryl group are directly connected by a covalent bond. The description of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups here may refer to the description of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups in the part of the above general formula (I), and details are not repeated here.

It will be noted that in a case where R₃ is selected from substituted C1 to C39 alkyl groups, substituted C2 to C39 alkenyl groups, substituted C2 to C39 alkynyl groups, substituted C6 to C39 aryl groups, substituted C5 to C60 heteroaryl groups, substituted C6 to C60 aryloxy groups, substituted C1 to C39 alkoxy groups, substituted C6 to C39 arylamine groups, substituted C3 to C39 cycloalkyl groups, substituted C3 to C39 heterocyclylalkyl groups, substituted C1 to C39 alkylsilyl groups, substituted C1 to C39 alkyl boryl groups, substituted C6 to C39 aryl boryl groups, substituted C6 to C39 arylphosphino groups, and substituted C6 to C39 arylsilyl groups, the type of the substituent is not limited here.

The connection position between L₃ and the phenanthryl group shown in the general formula (II) refers to that L₃ may be connected to any of carbon 1″, 2″, 3″, 4″, 5″, 6″, 7″, 8″, 9″ and 10″ in the IA-1 part, that is, L₃ may be connected to any of carbon 1″, 2″, 3″, 4″, 5″, 6″, 7″, 8″, 9″ and 10″ that has a substitution position.

The connection position between R₃ and the phenanthryl group shown in the general formula (II) refers to that R₃ may be connected to any of carbon 1″, 2″, 3″, 4″, 5″, 6″, 7″, 8″, 9″ and 10″ in the IA-1 part, that is, R₃ may be connected to any of carbon 1″, 2″, 3″, 4″, 5″, 6″, 7″, 8″, 9″ and 10″ that has a substitution position.

In a case where the value of m is greater than or equal to 2, that is, in a case where the number of R₂ is greater than or equal to 2, the types of the m R₃ may be the same or different.

It can be understood that in a case where a fused ring aryl group in the first substituent unit IA in the structure represented by the general formula (I) contains a phenanthryl group, the structure represented by the general formula (I) may be transformed into the structure represented by the general formula (II). It will be noted that the description of “containing a phenanthryl group” here means that the fused ring aryl group is a phenanthryl group, or means that the fused ring aryl group is another fused ring aryl group formed by further fusing on a phenanthryl group.

Based on the structure represented by the general formula (II), the fused ring aryl group in the first substitution unit IA of the functional layer material F contains a phenanthryl group, and the phenanthryl group is a structure formed by three six-membered carbon rings fused together and has a large conjugated area, and may produce a good conjugated effect. In this way, due to a synergistic effect of the second substitution unit IB and the third substitution unit IC, the light-emitting device 100 may have a relatively high luminous efficiency, a relatively low voltage and a long service life.

In some embodiments, X is oxygen and Y is a single bond. That is, the functional layer material F is selected from any of structures represented by a following general formula (V).

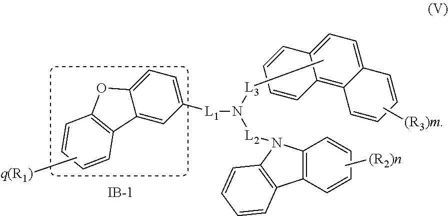

In a case where X is oxygen and Y is a single bond, the IB part in the general formula (II) is transformed into the IB-1 part in the general formula (V). The IB-1 part in the general formula (V) contains a dibenzofuran structure. The heteroatom oxygen in the dibenzofuran structure may improve a hole transport ability of the functional layer material F, increase the amount of holes transported to the light-emitting layer 131, improve a recombination probability of holes and electrons in the light-emitting layer 131, thereby improving an emission efficiency. Moreover, since the dibenzofuran structure has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131. In this way, the luminous efficiency of the light-emitting device 100 may be effectively improved.

An exemplary structure of the functional layer material F in the structure represented by the general formula (V) is described below.

In some examples, in a case where L₁ is a single bond, L₂ is a phenylene group, and L₃ is a single bond, the structural formula of the functional layer material F may be as shown below.

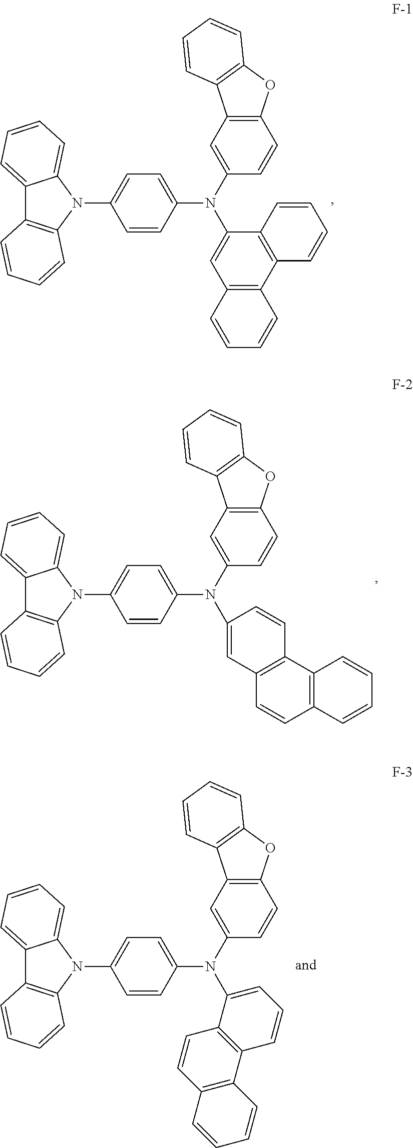 [SMILES: c1ccc2c(c1)cc(N(c1ccc(-n3c4ccccc4c4ccccc43)cc1)c1ccc3oc4ccccc4c3c1)c1ccccc12 ; c1ccc2c(c1)ccc1cc(N(c3ccc(-n4c5ccccc5c5ccccc54)cc3)c3ccc4oc5ccccc5c4c3)ccc12 ; c1ccc2c(c1)ccc1c(N(c3ccc(-n4c5ccccc5c5ccccc54)cc3)c3ccc4oc5ccccc5c4c3)cccc12]

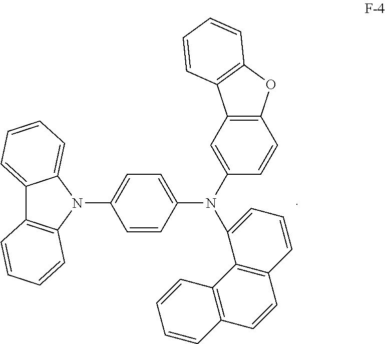 [SMILES: c1ccc2c(c1)ccc1cccc(N(c3ccc(-n4c5ccccc5c5ccccc54)cc3)c3ccc4oc5ccccc5c4c3)c12]

In some examples, in a case where L₁ is a single bond, L₂ is a biphenylene group, and L₃ is a single bond, the structural formula of the functional layer material F may be as shown below.

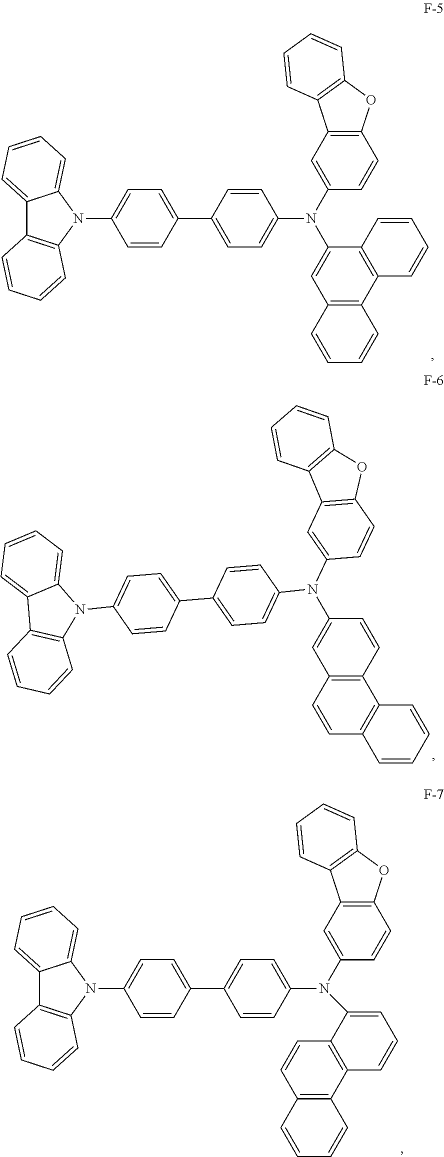 [SMILES: c1ccc2c(c1)cc(N(c1ccc(-c3ccc(-n4c5ccccc5c5ccccc54)cc3)cc1)c1ccc3oc4ccccc4c3c1)c1ccccc12 ; c1ccc2c(c1)ccc1cc(N(c3ccc(-c4ccc(-n5c6ccccc6c6ccccc65)cc4)cc3)c3ccc4oc5ccccc5c4c3)ccc12 ; c1ccc2c(c1)ccc1c(N(c3ccc(-c4ccc(-n5c6ccccc6c6ccccc65)cc4)cc3)c3ccc4oc5ccccc5c4c3)cccc12]

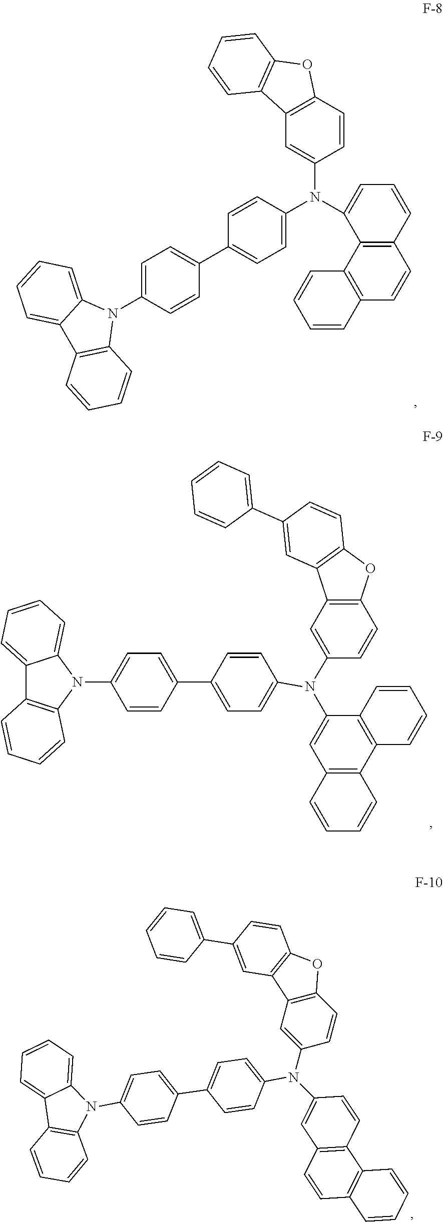 [SMILES: c1ccc2c(c1)ccc1cccc(N(c3ccc(-c4ccc(-n5c6ccccc6c6ccccc65)cc4)cc3)c3ccc4oc5ccccc5c4c3)c12 ; c1ccc(-c2ccc3oc4ccc(N(c5ccc(-c6ccc(-n7c8ccccc8c8ccccc87)cc6)cc5)c5cc6ccccc6c6ccccc56)cc4c3c2)cc1 ; c1ccc(-c2ccc3oc4ccc(N(c5ccc(-c6ccc(-n7c8ccccc8c8ccccc87)cc6)cc5)c5ccc6c(ccc7ccccc76)c5)cc4c3c2)cc1]

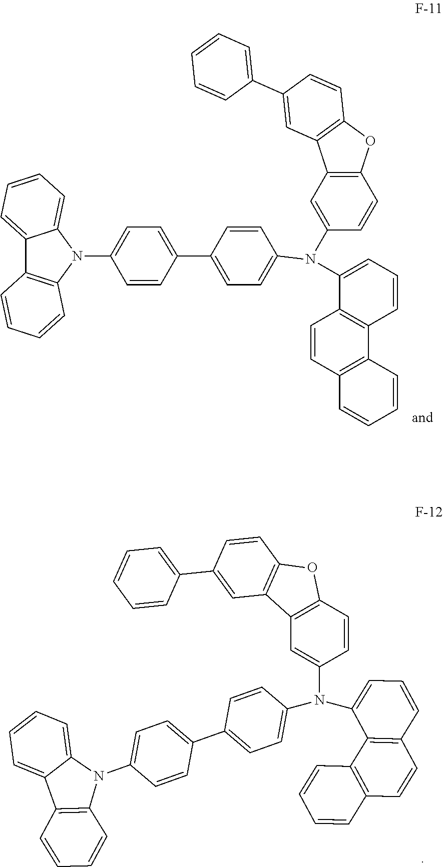 [SMILES: c1ccc(-c2ccc3oc4ccc(N(c5ccc(-c6ccc(-n7c8ccccc8c8ccccc87)cc6)cc5)c5cccc6c5ccc5ccccc56)cc4c3c2)cc1 ; c1ccc(-c2ccc3oc4ccc(N(c5ccc(-c6ccc(-n7c8ccccc8c8ccccc87)cc6)cc5)c5cccc6ccc7ccccc7c56)cc4c3c2)cc1]

In some examples, in a case where L₁ is a single bond, L₂ is a phenylene group, and L₃ is phenylene group, the structural formula of the functional layer material F may be as shown below.

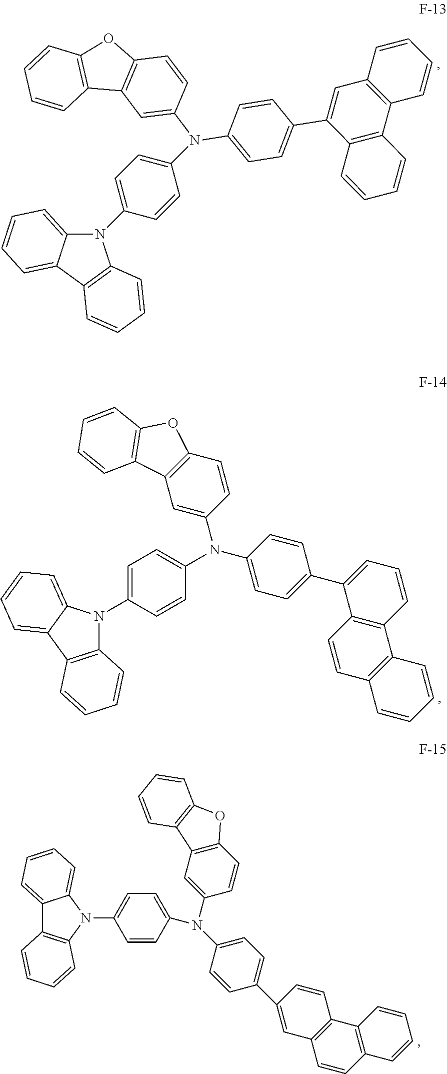 [SMILES: c1ccc2c(c1)cc(-c1ccc(N(c3ccc(-n4c5ccccc5c5ccccc54)cc3)c3ccc4oc5ccccc5c4c3)cc1)c1ccccc12 ; c1ccc2c(c1)ccc1c(-c3ccc(N(c4ccc(-n5c6ccccc6c6ccccc65)cc4)c4ccc5oc6ccccc6c5c4)cc3)cccc12 ; c1ccc2c(c1)ccc1cc(-c3ccc(N(c4ccc(-n5c6ccccc6c6ccccc65)cc4)c4ccc5oc6ccccc6c5c4)cc3)ccc12]

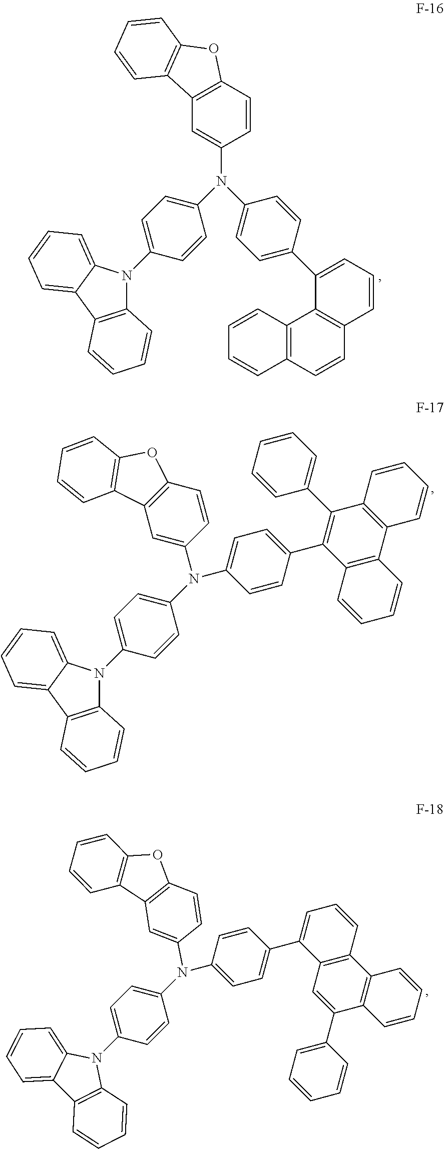 [SMILES: c1ccc2c(c1)ccc1cccc(-c3ccc(N(c4ccc(-n5c6ccccc6c6ccccc65)cc4)c4ccc5oc6ccccc6c5c4)cc3)c12 ; c1ccc(-c2c(-c3ccc(N(c4ccc(-n5c6ccccc6c6ccccc65)cc4)c4ccc5oc6ccccc6c5c4)cc3)c3ccccc3c3ccccc23)cc1 ; c1ccc(-c2cc3c(-c4ccc(N(c5ccc(-n6c7ccccc7c7ccccc76)cc5)c5ccc6oc7ccccc7c6c5)cc4)cccc3c3ccccc23)cc1]

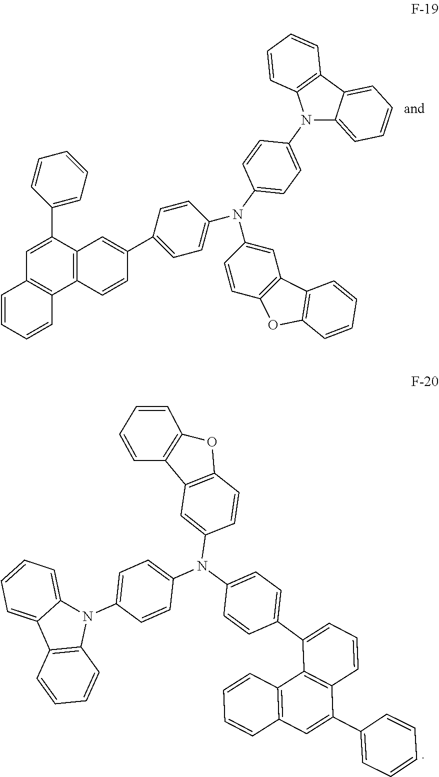 [SMILES: c1ccc(-c2cc3ccccc3c3ccc(-c4ccc(N(c5ccc(-n6c7ccccc7c7ccccc76)cc5)c5ccc6oc7ccccc7c6c5)cc4)cc23)cc1 ; c1ccc(-c2cc3ccccc3c3c(-c4ccc(N(c5ccc(-n6c7ccccc7c7ccccc76)cc5)c5ccc6oc7ccccc7c6c5)cc4)cccc23)cc1]

In some examples, in a case where L₁ is a phenylene group, L₂ is a phenylene group, and L₃ is a single bond, the structural formula of the functional layer material F may be as shown below.

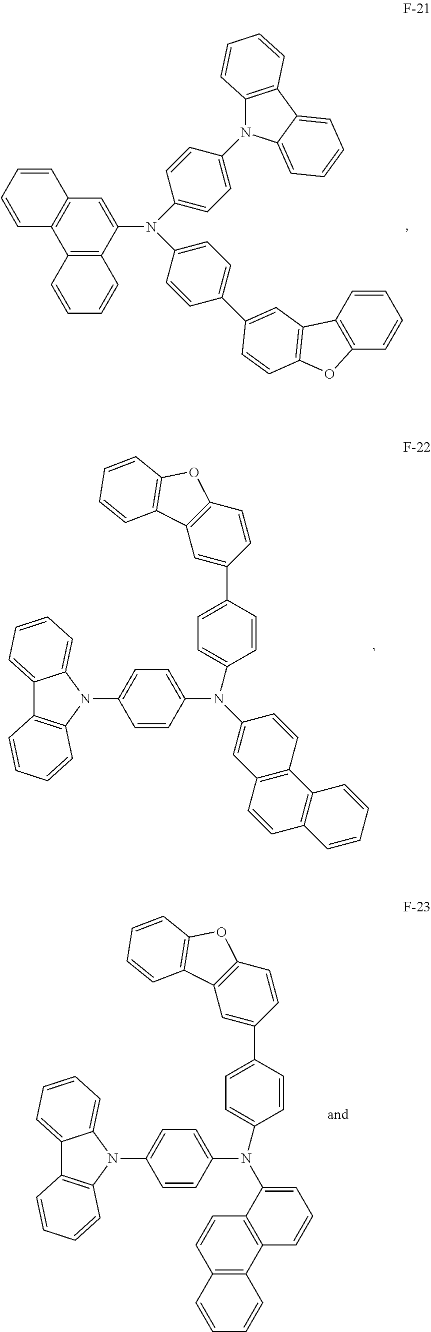 [SMILES: c1ccc2c(c1)cc(N(c1ccc(-c3ccc4oc5ccccc5c4c3)cc1)c1ccc(-n3c4ccccc4c4ccccc43)cc1)c1ccccc12 ; c1ccc2c(c1)ccc1cc(N(c3ccc(-c4ccc5oc6ccccc6c5c4)cc3)c3ccc(-n4c5ccccc5c5ccccc54)cc3)ccc12 ; c1ccc2c(c1)ccc1c(N(c3ccc(-c4ccc5oc6ccccc6c5c4)cc3)c3ccc(-n4c5ccccc5c5ccccc54)cc3)cccc12]

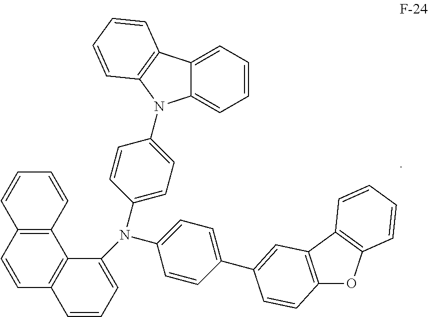 [SMILES: c1ccc2c(c1)ccc1cccc(N(c3ccc(-c4ccc5oc6ccccc6c5c4)cc3)c3ccc(-n4c5ccccc5c5ccccc54)cc3)c12]

In some examples, in a case where L₁ is a phenylene group, L₂ is a naphthylene group, and L₃ is a single bond, the structural formula of the functional layer material F may be as shown below.

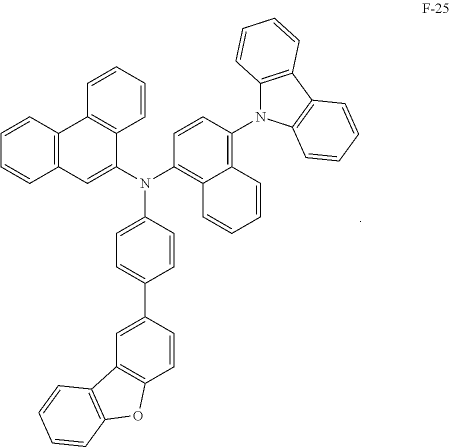 [SMILES: c1ccc2c(c1)cc(N(c1ccc(-c3ccc4oc5ccccc5c4c3)cc1)c1ccc(-n3c4ccccc4c4ccccc43)c3ccccc13)c1ccccc12]

In some examples, in a case where L₁ is a phenylene group, L₂ is a biphenylene group, and L₃ is a single bond, the structural formula of the functional layer material F may be as shown below.

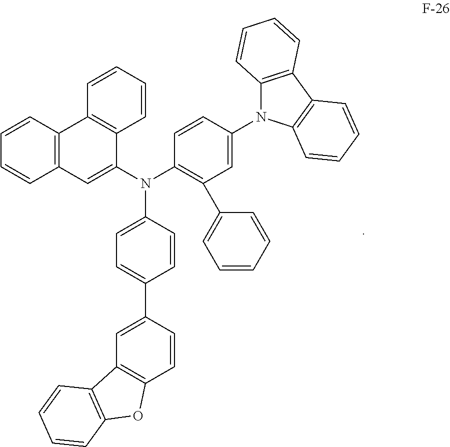 [SMILES: c1ccc(-c2cc(-n3c4ccccc4c4ccccc43)ccc2N(c2ccc(-c3ccc4oc5ccccc5c4c3)cc2)c2cc3ccccc3c3ccccc23)cc1]

In some examples, in a case where L₁ is a phenylene group, L₂ is a phenylene group, and L₃ is a phenylene group, the structural formula of the functional layer material F may be as shown below.

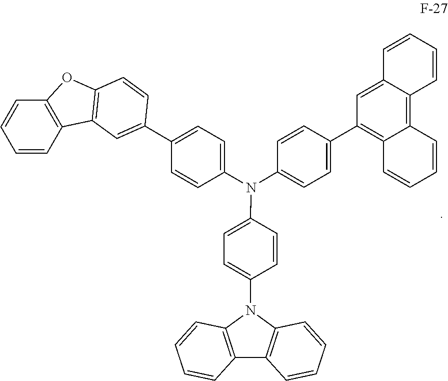 [SMILES: c1ccc2c(c1)cc(-c1ccc(N(c3ccc(-c4ccc5oc6ccccc6c5c4)cc3)c3ccc(-n4c5ccccc5c5ccccc54)cc3)cc1)c1ccccc12]

It will be noted that the structural formulas listed above are examples of the structure of the functional layer material F, but not limitations on the functional layer material F. In addition, F-x in the above structural formulas is an antonomasia of each structural formula, and is not a part of structure of the structural formula, where x is a positive integer.

In some embodiments, X is sulfur and Y is a single bond. That is, the functional layer material F is selected from any of structures represented by a following general formula (VI).

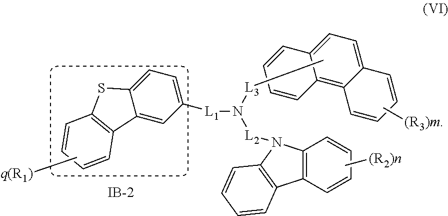

In a case where X is sulfur and Y is a single bond, the IB part in the general formula (II) is transformed into the IB-2 part in the general formula (VI). The IB-2 part in the general formula (VI) contains a dibenzothiophene structure. The heteroatom sulfur in the dibenzothiophene structure may improve a hole transport ability of the functional layer material F, increase the amount of holes transported to the light-emitting layer 131, improve a recombination probability of holes and electrons in the light-emitting layer 131, thereby improving an emission efficiency. Moreover, since the dibenzothiophene structure has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131. In this way, the luminous efficiency of the light-emitting device 100 may be effectively improved.

An exemplary structure of the functional layer material F in the structure represented by the general formula (VI) is described below.

In some examples, in a case where L₁ is a single bond, L₂ is a phenylene group, and L₃ is a single bond, the structural formula of the functional layer material F may be as shown below.

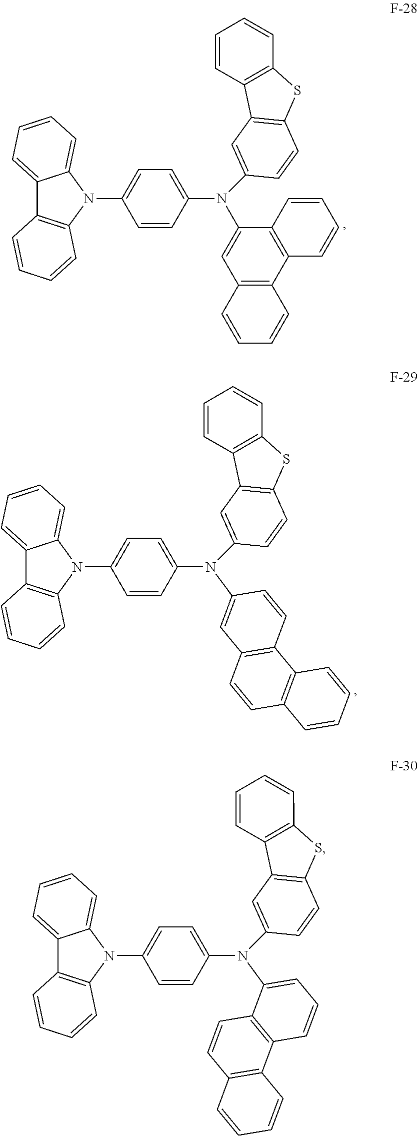

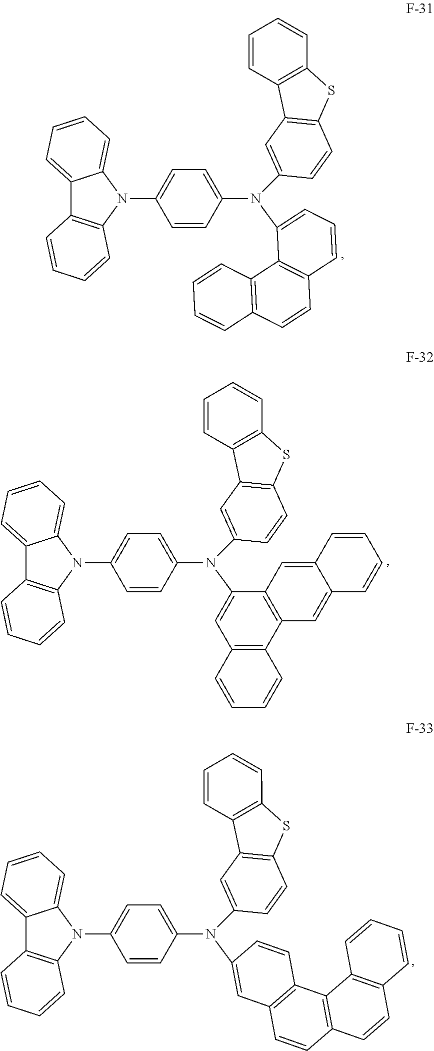

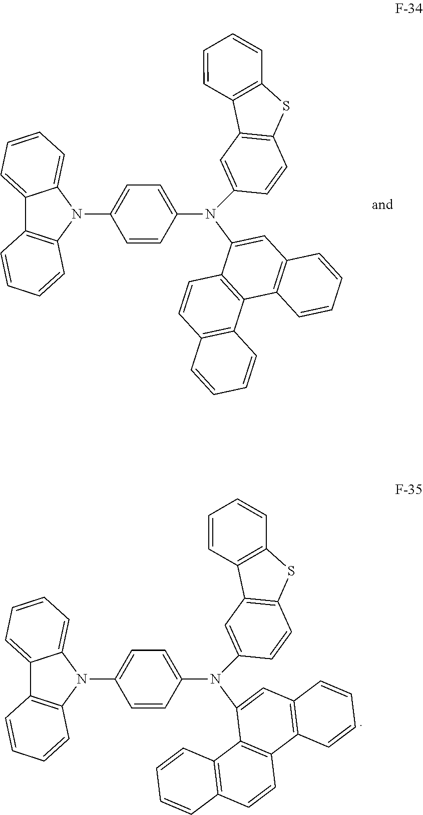 [SMILES: c1ccc2c(c1)ccc1c(N(c3ccc(-n4c5ccccc5c5ccccc54)cc3)c3ccc4sc5ccccc5c4c3)cc3ccccc3c12 ; c1ccc2c(c1)cc(N(c1ccc(-n3c4ccccc4c4ccccc43)cc1)c1ccc3sc4ccccc4c3c1)c1c3ccccc3ccc21]

In some examples, in a case where L₁ is a single bond, L₂ is a biphenylene group, and L₃ is a single bond, the structural formula of the functional layer material F may be as shown below.

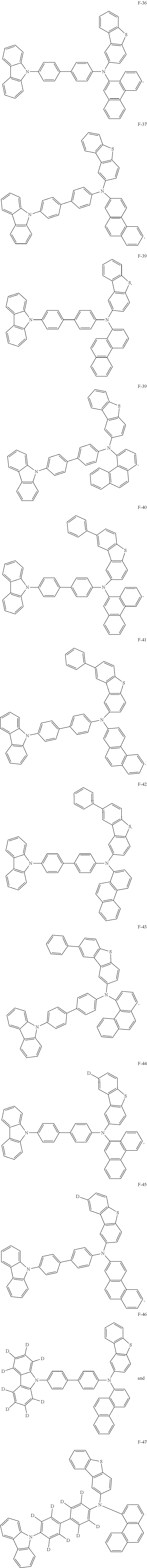

In some examples, in a case where L₁ is a single bond, L₂ is a phenylene group, and L₃ is phenylene group, the structural formula of the functional layer material F may be as shown below.

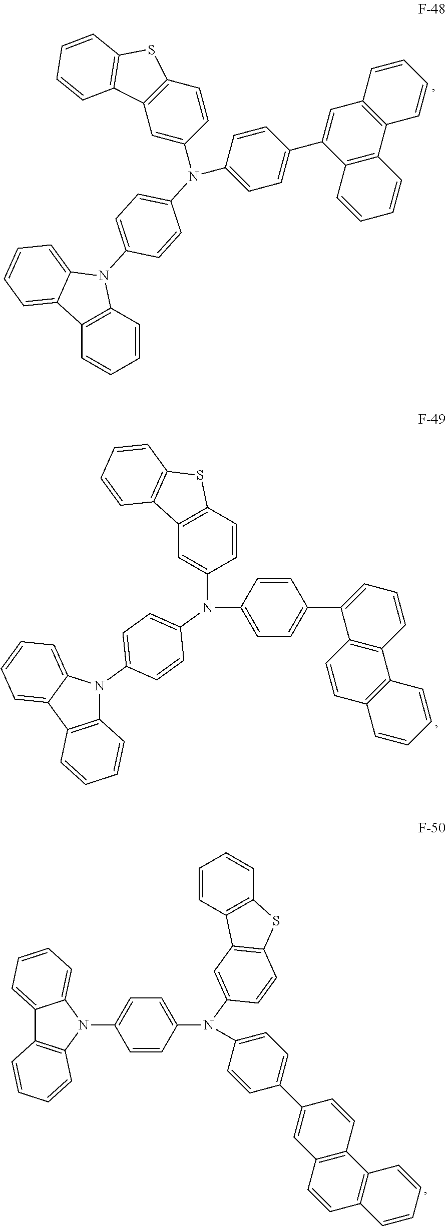 [SMILES: c1ccc2c(c1)cc(-c1ccc(N(c3ccc(-n4c5ccccc5c5ccccc54)cc3)c3ccc4sc5ccccc5c4c3)cc1)c1ccccc12 ; c1ccc2c(c1)ccc1c(-c3ccc(N(c4ccc(-n5c6ccccc6c6ccccc65)cc4)c4ccc5sc6ccccc6c5c4)cc3)cccc12 ; c1ccc2c(c1)ccc1cc(-c3ccc(N(c4ccc(-n5c6ccccc6c6ccccc65)cc4)c4ccc5sc6ccccc6c5c4)cc3)ccc12]

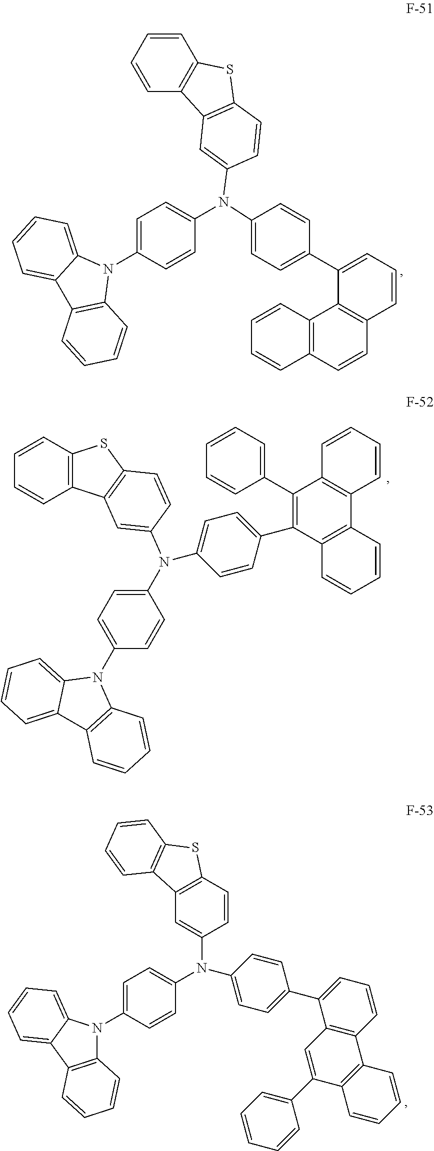 [SMILES: c1ccc2c(c1)ccc1cccc(-c3ccc(N(c4ccc(-n5c6ccccc6c6ccccc65)cc4)c4ccc5sc6ccccc6c5c4)cc3)c12 ; c1ccc(-c2c(-c3ccc(N(c4ccc(-n5c6ccccc6c6ccccc65)cc4)c4ccc5sc6ccccc6c5c4)cc3)c3ccccc3c3ccccc23)cc1 ; c1ccc(-c2cc3c(-c4ccc(N(c5ccc(-n6c7ccccc7c7ccccc76)cc5)c5ccc6sc7ccccc7c6c5)cc4)cccc3c3ccccc23)cc1]

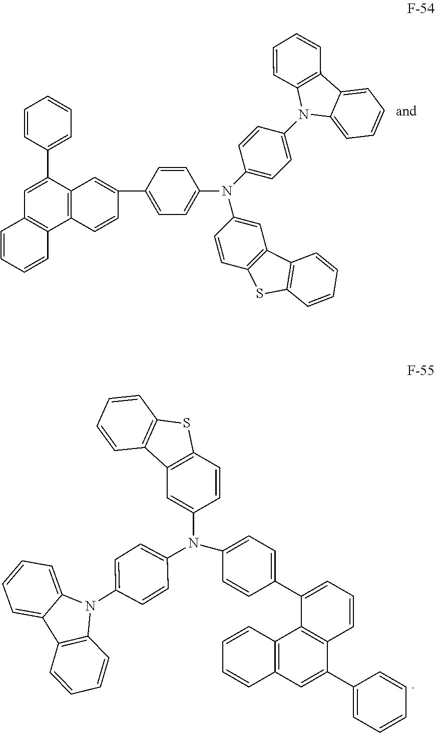 [SMILES: c1ccc(-c2cc3ccccc3c3ccc(-c4ccc(N(c5ccc(-n6c7ccccc7c7ccccc76)cc5)c5ccc6sc7ccccc7c6c5)cc4)cc23)cc1 ; c1ccc(-c2cc3ccccc3c3c(-c4ccc(N(c5ccc(-n6c7ccccc7c7ccccc76)cc5)c5ccc6sc7ccccc7c6c5)cc4)cccc23)cc1]

In some examples, in a case where L₁ is a single bond, L₂ is a biphenylene group, and L₃ is a phenylene group, the structural formula of the functional layer material F may be as shown below.

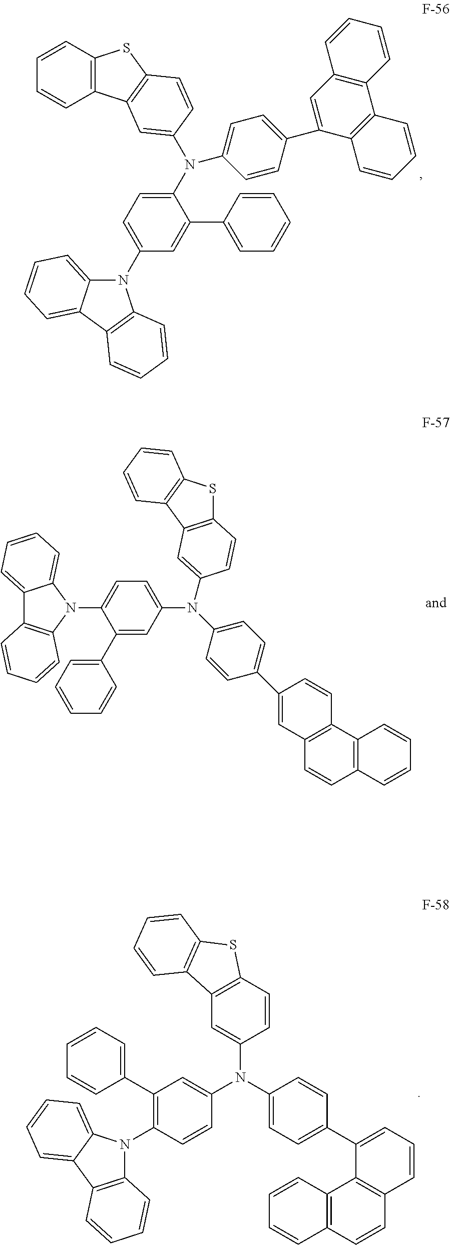 [SMILES: c1ccc(-c2cc(-n3c4ccccc4c4ccccc43)ccc2N(c2ccc(-c3cc4ccccc4c4ccccc34)cc2)c2ccc3sc4ccccc4c3c2)cc1 ; c1ccc(-c2cc(N(c3ccc(-c4ccc5c(ccc6ccccc65)c4)cc3)c3ccc4sc5ccccc5c4c3)ccc2-n2c3ccccc3c3ccccc32)cc1 ; c1ccc(-c2cc(N(c3ccc(-c4cccc5ccc6ccccc6c45)cc3)c3ccc4sc5ccccc5c4c3)ccc2-n2c3ccccc3c3ccccc32)cc1]

In some examples, in a case where L₁ is a single bond, L₂ is a phenylene group, and L₃ is a biphenylene group, the structural formula of the functional layer material F may be as shown below.

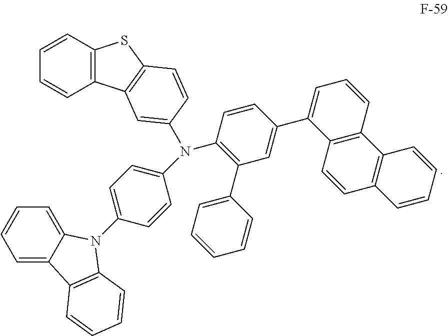 [SMILES: c1ccc(-c2cc(-c3cccc4c3ccc3ccccc34)ccc2N(c2ccc(-n3c4ccccc4c4ccccc43)cc2)c2ccc3sc4ccccc4c3c2)cc1]

In some examples, in a case where L₁ is a phenylene group, L₂ is a phenylene group, and L₃ is a single bond, the structural formula of the functional layer material F may be as shown below.

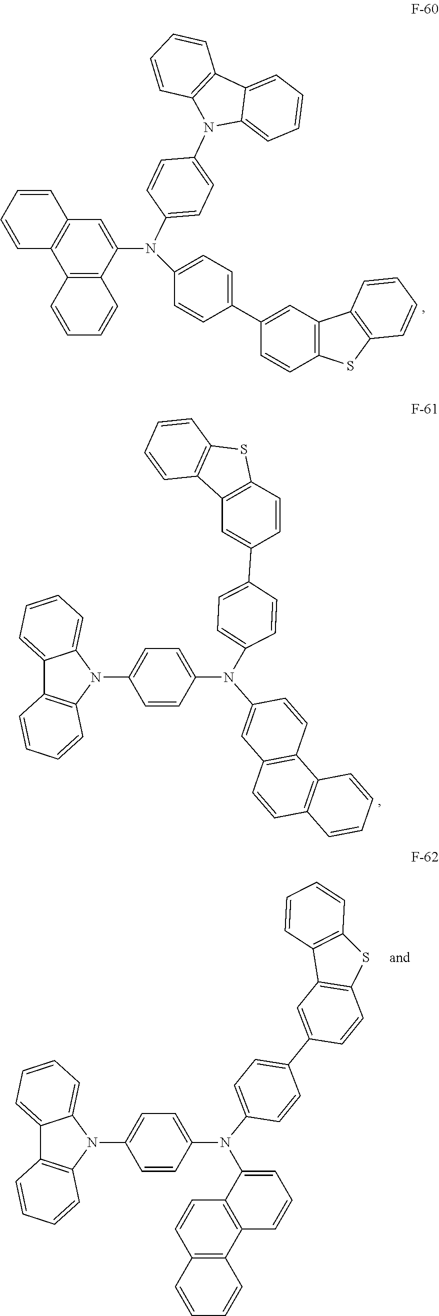 [SMILES: c1ccc2c(c1)cc(N(c1ccc(-c3ccc4sc5ccccc5c4c3)cc1)c1ccc(-n3c4ccccc4c4ccccc43)cc1)c1ccccc12 ; c1ccc2c(c1)ccc1cc(N(c3ccc(-c4ccc5sc6ccccc6c5c4)cc3)c3ccc(-n4c5ccccc5c5ccccc54)cc3)ccc12 ; c1ccc2c(c1)ccc1c(N(c3ccc(-c4ccc5sc6ccccc6c5c4)cc3)c3ccc(-n4c5ccccc5c5ccccc54)cc3)cccc12]

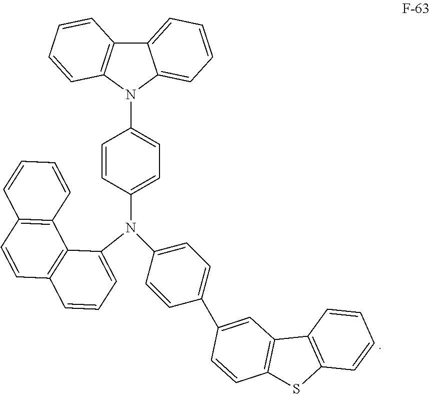 [SMILES: c1ccc2c(c1)ccc1cccc(N(c3ccc(-c4ccc5sc6ccccc6c5c4)cc3)c3ccc(-n4c5ccccc5c5ccccc54)cc3)c12]

It will be noted that the structural formulas listed above are examples of the structure of the functional layer material F, but not limitations on the functional layer material F. In addition, F-x in the above structural formulas is an antonomasia of each structural formula, and is not a part of structure of the structural formula, where x is a positive integer.

In some embodiments, X is oxygen and Y is oxygen. That is, the functional layer material F is selected from any of structures represented by a following general formula (VII).

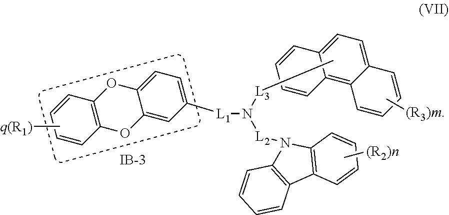

In a case where X is oxygen and Y is oxygen, the IB part in the general formula (II) is transformed into the IB-3 part in the general formula (VII). The IB-3 part in the general formula (VII) contains a dibenzo[b,e][1,4]dioxin structure. The heteroatom oxygen in the dibenzo[b,e][1,4]dioxin structure may improve a hole transport ability of the functional layer material F, increase the amount of holes transported to the light-emitting layer 131, improve a recombination probability of holes and electrons in the light-emitting layer 131, thereby improving an emission efficiency. Moreover, since the dibenzo[b,e][1,4]dioxin structure has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131. In this way, the luminous efficiency of the light-emitting device 100 may be effectively improved.

An exemplary structure of the functional layer material F in the structure represented by the general formula (VII) is described below.

In some examples, in a case where L₁ is a single bond, L₂ is a phenylene group, and L₃ is a single bond, the structural formula of the functional layer material F may be as shown below.

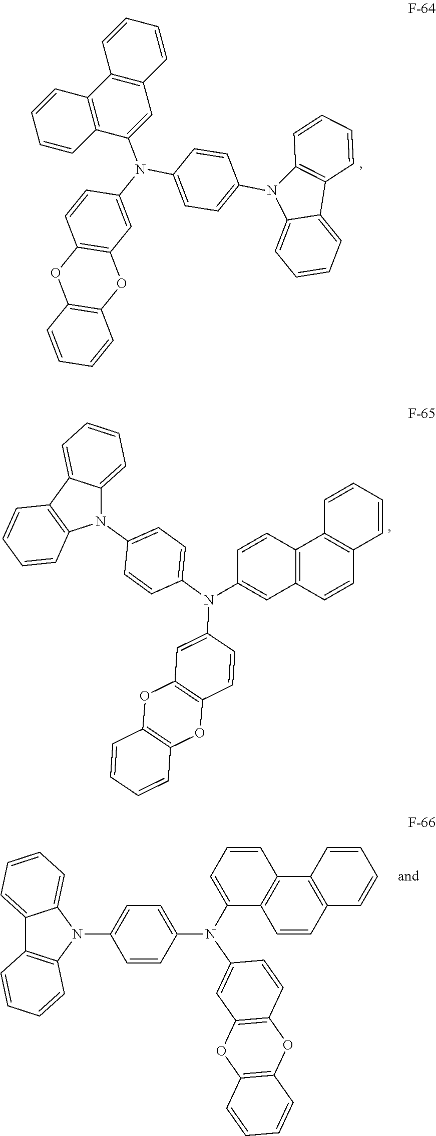 [SMILES: c1ccc2c(c1)Oc1ccc(N(c3ccc(-n4c5ccccc5c5ccccc54)cc3)c3cc4ccccc4c4ccccc34)cc1O2 ; c1ccc2c(c1)Oc1ccc(N(c3ccc(-n4c5ccccc5c5ccccc54)cc3)c3ccc4c(ccc5ccccc54)c3)cc1O2 ; c1ccc2c(c1)Oc1ccc(N(c3ccc(-n4c5ccccc5c5ccccc54)cc3)c3cccc4c3ccc3ccccc34)cc1O2]

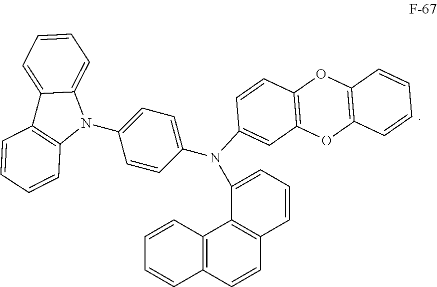 [SMILES: c1ccc2c(c1)Oc1ccc(N(c3ccc(-n4c5ccccc5c5ccccc54)cc3)c3cccc4ccc5ccccc5c34)cc1O2]

It will be noted that the structural formulas listed above are examples of the structure of the functional layer material F, but not limitations on the functional layer material F. In addition, F-x in the above structural formulas is an antonomasia of each structural formula, and is not a part of structure of the structural formula, where x is a positive integer.

In some embodiments, X is oxygen and Y is sulfur. That is, the functional layer material F is selected from any of structures represented by a following general formula (VIII).

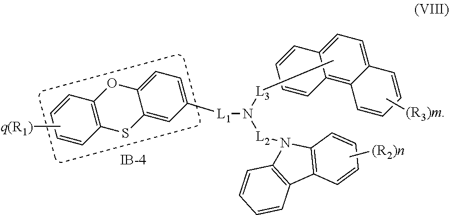

In a case where X is oxygen and Y is sulfur, the IB part in the general formula (II) is transformed into the IB-4 part in the general formula (VIII). The IB-4 part in the general formula (VIII) contains a phenoxathiine structure (i.e., a phenoxathiin structure). The heteroatoms oxygen and sulfur in the phenoxathiine structure may improve a hole transport ability of the functional layer material F, increase the amount of holes transported to the light-emitting layer 131, improve a recombination probability of holes and electrons in the light-emitting layer 131, thereby improving an emission efficiency. Moreover, since the phenoxathiine structure has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131. In this way, the luminous efficiency of the light-emitting device 100 may be effectively improved.

An exemplary structure of the functional layer material F in the structure represented by the general formula (VIII) is described below.

In some examples, in a case where L₁ is a single bond, L₂ is a phenylene group, and L₃ is phenylene group, the structural formula of the functional layer material F may be as shown below.

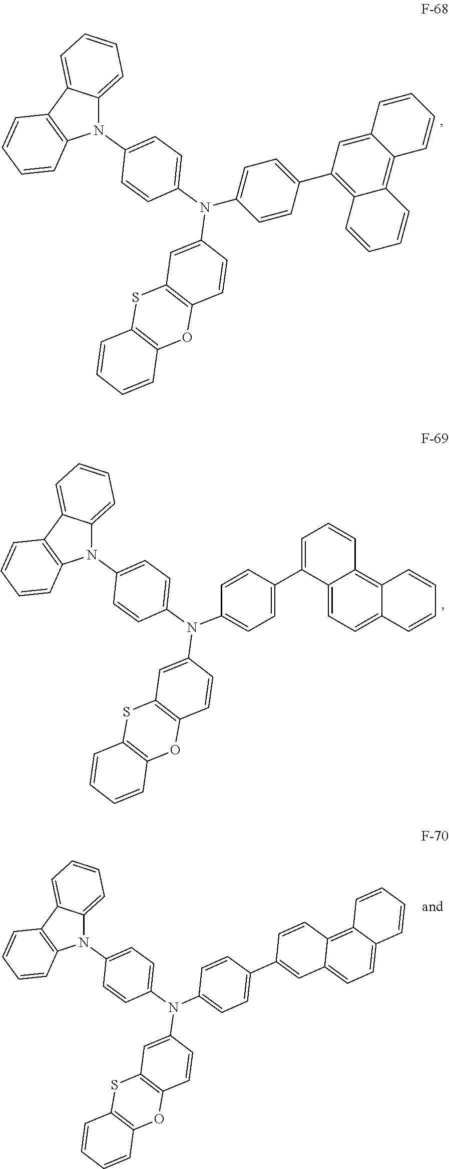 [SMILES: c1ccc2c(c1)Oc1ccc(N(c3ccc(-c4cc5ccccc5c5ccccc45)cc3)c3ccc(-n4c5ccccc5c5ccccc54)cc3)cc1S2 ; c1ccc2c(c1)Oc1ccc(N(c3ccc(-c4cccc5c4ccc4ccccc45)cc3)c3ccc(-n4c5ccccc5c5ccccc54)cc3)cc1S2 ; c1ccc2c(c1)Oc1ccc(N(c3ccc(-c4ccc5c(ccc6ccccc65)c4)cc3)c3ccc(-n4c5ccccc5c5ccccc54)cc3)cc1S2]

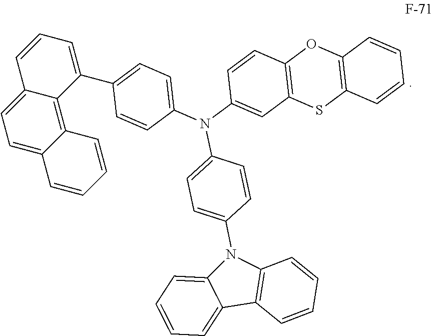 [SMILES: c1ccc2c(c1)Oc1ccc(N(c3ccc(-c4cccc5ccc6ccccc6c45)cc3)c3ccc(-n4c5ccccc5c5ccccc54)cc3)cc1S2]

It will be noted that the structural formulas listed above are examples of the structure of the functional layer material F, but not limitations on the functional layer material F. In addition, F-x in the above structural formulas is an antonomasia of each structural formula, and is not a part of structure of the structural formula, where x is a positive integer.

In some embodiments, the functional layer material F is selected from any of structures represented by a general formula (III).

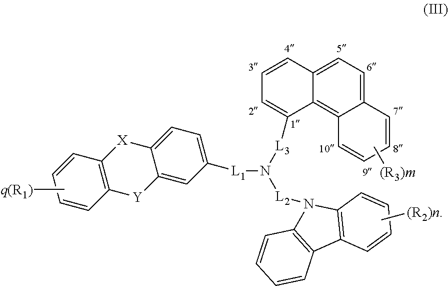

It can be understood that in a case where L₃ is connected to carbon 1″ in the IA-1 part (i.e., the phenanthryl group) in the structure represented by the general formula (II), the structure represented by the general formula (II) may be transformed into the structure represented by the general formula (III). It will be noted that since the structure of the phenanthryl group is a symmetrical structure, in a case where R₃ is hydrogen, the structure in a case of L₃ being connected to carbon 1″ in the IA-1 part and the structure in a case of L₃ being connected to carbon 10″ in the IA-1 part are of the same structure.

An exemplary structure of the functional layer material F in the structure represented by the general formula (III) is described below.

In some examples, in a case where X is oxygen and Y is a single bond, the structural formula of the functional layer material F may be shown as (F-4), (F-8), (F-12), (F-16), (F-20) and (F-24) above.

In some examples, in a case where X is sulfur and Y is a single bond, the structural formula of the functional layer material F may be shown as (F-31), (F-35), (F-39), (F-43), (F-47), (F-51), (F-55), (F-58) and (F-63) above.

In some examples, in a case where X is oxygen and Y is oxygen, the structural formula of the functional layer material F may be shown as (F-67) above.

In some examples, in a case where X is oxygen and Y is sulfur, the structural formula of the functional layer material F may be shown as (F-71) above.

It will be noted that the structural formulas listed above are examples of the structure of the functional layer material F, but not limitations on the functional layer material F.

In some embodiments, the functional layer material F is selected from any of structures represented by a general formula (IV).

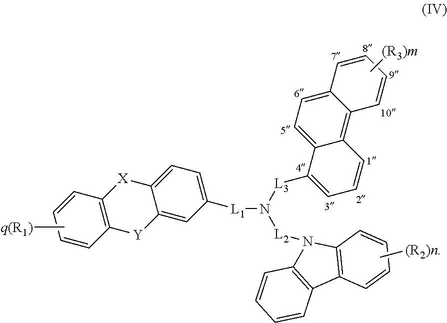

It can be understood that in a case where L₃ is connected to carbon 4″ in the IA-1 part (i.e., the phenanthryl group) in the structure represented by the general formula (II), the structure represented by the general formula (II) may be transformed into the structure represented by the general formula (IV). It will be noted that since the structure of the phenanthryl group is a symmetrical structure, in a case where R₃ is hydrogen, the structure in a case of L₃ being connected to carbon 4″ in the IA-1 part and the structure in a case of L₃ being connected to carbon 7″ in the IA-1 part are of the same structure.

An exemplary structure of the functional layer material F in the structure represented by the general formula (IV) is described below.

In some examples, in a case where X is oxygen and Y is a single bond, the structural formula of the functional layer material F may be shown as (F-3), (F-7), (F-11), (F-14), (F-18) and (F-23) above.

In some examples, in a case where X is sulfur and Y is a single bond, the structural formula of the functional layer material F may be shown as (F-30), (F-34), (F-38), (F-42), (F-46), (F-53), (F-49), (F-59) and (F-62) above.

In some examples, in a case where X is oxygen and Y is oxygen, the structural formula of the functional layer material F may be shown as (F-66) above.

In some examples, in a case where X is oxygen and Y is sulfur, the structural formula of the functional layer material F may be shown as (F-69) above.

It will be noted that the structural formulas listed above are examples of the structure of the functional layer material F, but not limitations on the functional layer material F.

In some embodiments, the functional layer material F is selected from any of structures represented by a general formula (VIIII).

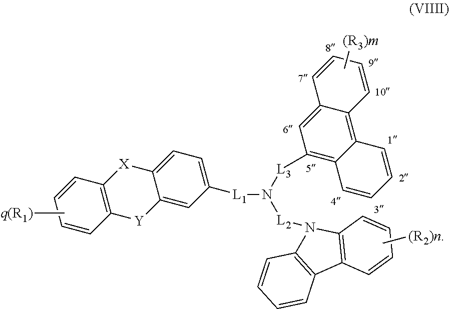

It can be understood that in a case where L₃ is connected to carbon 5″ in the IA-1 part (i.e., the phenanthryl group) in the structure represented by the general formula (II), the structure represented by the general formula (II) may be transformed into the structure represented by the general formula (VIIII). It will be noted that since the structure of the phenanthryl group is a symmetrical structure, in a case where R₃ is hydrogen, the structure in a case of L₃ being connected to carbon 5″ in the IA-1 part and the structure in a case of L₃ being connected to carbon 6″ in the IA-1 part are of the same structure.

An exemplary structure of the functional layer material F in the structure represented by the general formula (VIIII) is described below.

In some examples, in a case where X is oxygen and Y is a single bond, the structural formula of the functional layer material F may be shown as (F-1), (F-5), (F-9), (F-13), (F-17), (F-21), (F-25), (F-26) and (F-27) above.

In some examples, in a case where X is sulfur and Y is a single bond, the structural formula of the functional layer material F may be shown as (F-28), (F-32), (F-36), (F-40), (F-44), (F-48), (F-52), (F-56) and (F-60) above.

In some examples, in a case where X is oxygen and Y is oxygen, the structural formula of the functional layer material F may be shown as (F-64) above.

In some examples, in a case where X is oxygen and Y is sulfur, the structural formula of the functional layer material F may be shown as (F-68) above.

It will be noted that the structural formulas listed above are examples of the structure of the functional layer material F, but not limitations on the functional layer material F.

In some embodiments, the functional layer material F is selected from any of structures represented by a general formula (X).

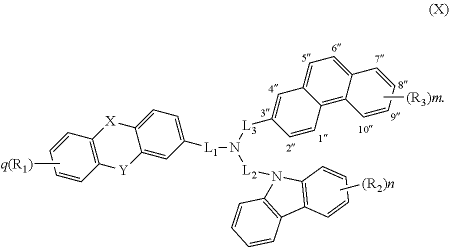

It can be understood that in a case where L₃ is connected to carbon 3″ in the IA-1 part (i.e., the phenanthryl group) in the structure represented by the general formula (II), the structure represented by the general formula (II) may be transformed into the structure represented by the general formula (X). It will be noted that since the structure of the phenanthryl group is a symmetrical structure, in a case where R₃ is hydrogen, the structure in a case of L₃ being connected to carbon 3″ in the IA-1 part and the structure in a case of L₃ being connected to carbon 8″ in the IA-1 part are of the same structure.

An exemplary structure of the functional layer material F in the structure represented by the general formula (X) is described below.

In some examples, in a case where X is oxygen and Y is a single bond, the structural formula of the functional layer material F may be shown as (F-2), (F-6), (F-10), (F-15), (F-19) and (F-22) above.

In some examples, in a case where X is sulfur and Y is a single bond, the structural formula of the functional layer material F may be shown as (F-29), (F-33), (F-37), (F-41), (F-45), (F-50), (F-54), (F-57) and (F-61) above.

In some examples, in a case where X is oxygen and Y is oxygen, the structural formula of the functional layer material F may be shown as (F-65) above.

In some examples, in a case where X is oxygen and Y is sulfur, the structural formula of the functional layer material F may be shown as (F-70) above.

It will be noted that the structural formulas listed above are examples of the structure of the functional layer material F, but not limitations on the functional layer material F.

In order to improve a molecular stability of the functional layer material F, in some embodiments, in a case where the functional layer material F is selected from any of the structures represented by the general formula (I), the structure represented by the general formula (I) contains at least one deuterium; alternatively, in a case where the functional layer material F is selected from any of the structures represented by the general formula (II), the structure represented by the general formula (II) contains at least one deuterium; alternatively, in a case where the functional layer material F is selected from any of the structures represented by the general formula (III), the structure represented by the general formula (III) contains at least one deuterium; alternatively, in a case where the functional layer material F is selected from any of the structures represented by the general formula (IV), the structure represented by the general formula (IV) contains at least one deuterium.

In a case where the functional layer material F is selected from any of the structures represented by the general formula (V), the structure represented by the general formula (V) contains at least one deuterium; alternatively, in a case where the functional layer material F is selected from any of the structures represented by the general formula (VI), the structure represented by the general formula (VI) contains at least one deuterium; alternatively, in a case where the functional layer material F is selected from any of the structures represented by the general formula (VII), the structure represented by the general formula (VII) contains at least one deuterium; alternatively, in a case where the functional layer material F is selected from any of the structures represented by the general formula (VIII), the structure represented by the general formula (VIII) contains at least one deuterium; alternatively, in a case where the functional layer material F is selected from any of the structures represented by the general formula (VIIII), the structure represented by the general formula (VIIII) contains at least one deuterium; alternatively, in a case where the functional layer material F is selected from any of the structures represented by the general formula (X), the structure represented by the general formula (X) contains at least one deuterium.

Since an atomic mass unit of deuterium is twice an atomic mass unit of hydrogen, the setting of the structure shown in the above general formula containing at least one deuterium may allow physical properties of the functional layer material F to change. Specifically, after the functional layer material F has a substituent of deuterium, molecular vibration may be effectively suppressed, a bond length may be reduced, a bond energy may be enhanced, thereby improving the molecular stability. In this way, the stability of the functional layer material F may be improved, and the service life of the light-emitting device 100 may be prolonged.

The substitution position of deuterium will be described by considering an example of the structure represented by the general formula (II). The structure represented by the general formula (II) contains at least one deuterium, which means that the structure represented by the general formula (II) satisfies at least one of the following five conditions: (1) in the IA-1 part, a substituent of at least one carbon being deuterium or containing deuterium; (2) in a case where Y is selected from O and S, in the IB part, a substituent of at least one carbon in carbon 1 to carbon 12 being deuterium or containing deuterium; alternatively, in a case where Y is selected from N(R₄) and C(R₅R₆), in the IB part, a substituent of at least one of carbon 1 to carbon 12 and Y being deuterium or containing deuterium; (3) in the IC part, a substituent of at least one carbon being deuterium or containing deuterium; (4) a substituent of at least one of L₁, L₂ and L₃ being deuterium or containing deuterium; (5) at least one of R₁, R₂ and R₃ being deuterium or containing deuterium. That is, the substitution position of deuterium in the structure represented by the general formula (II) is not limited here. For understanding of containing at least one deuterium in other general formulas, reference may be made to the above contents, and details are not repeated here.

An exemplary structure of the functional layer material F in a case where the general formula (VI) contains at least one deuterium is introduced below.

In some examples, in a case where X is sulfur and Y is a single bond, the structural formula of the functional layer material F may be shown as (F-44), (F-45), (F-46) and (F-47) above.

It can be seen according to the functional layer material F shown in the above structural formulas (F-44), (F-45), (F-46) and (F-47) that in the structure shown in the general formula (VI), there is no limitation on the substitution position of deuterium. For example, the substitution position of deuterium may be located in the L₂ part, as shown in (F-47); alternatively, the substitution position of deuterium may be located in the R₂ part, as shown in (F-46); alternatively, the substitution position of deuterium may be located in the R₁ part, as shown in (F-44) and (F-45).

It will be noted that the structural formulas listed above are examples of the structure of the functional layer material F, but not limitations on the functional layer material F.

In some embodiments, in a case where m is greater than 2, two adjacent R₃ are capable of being bonded to form a ring.

It can be understood that in a first aspect, in a case where two adjacent R₃ are bonded to form a ring, in the IA-1 part in the structure represented by the general formula (II), the number of aromatic rings for forming a fused ring aryl group is greater than three, that is, at least one aromatic ring is fused with the phenanthryl group. In this way, the first substitution unit IA of the structure of the functional layer material F may have a large conjugated area and produce a good conjugated effect. Thus, due to a synergistic effect of the second substitution unit IB and the third substitution unit IC, the light-emitting device 100 may have a relatively high luminous efficiency, a relatively low voltage and a long service life. In a second aspect, in a case where two adjacent R₃ are bonded to form a ring, a portion connected to form a ring may increase an overall rigidity of the molecule of the functional layer material F, thereby having a rather high glass transition temperature Tg, increasing the stability of the material, and thus prolonging the life. In a third aspect, compared to a structure in which two adjacent R₃ exist but are not connected to form a ring, two adjacent R₃ being bonded to form a ring may appropriately reduce the three-dimensionality of the structure of the functional layer material F, thereby improving the mobility. In a fourth aspect, the setting of two adjacent R₃ being bonded to form a ring may increase richness of types of the functional layer material F, so that the functional layer material F may be combined with different types of electron transport materials to achieve carrier balance.

An exemplary structure of the functional layer material F in a case where two adjacent R₃ are bonded to form a ring is introduced below.

In some examples, in a case where X is sulfur and Y is a single bond, the structural formula of the functional layer material F may be shown as (F-32), (F-33), (F-34) and (F-35) above.

It can be seen according to the functional layer material F shown in the above structural formulas (F-32), (F-33), (F-34) and (F-35) that in the structure shown in the general formula (II), there is no limitations on the position at which two adjacent R₃ are bonded to form a ring. For example, the positions at which two adjacent R₃ are bonded to form a ring may be positions of carbon 2″ and carbon 3″, that is, two adjacent R₃ may form an aromatic ring with carbon 2″ and carbon 3″, as shown in (F-32). Alternatively, the positions at which two adjacent R₃ are bonded to form a ring may be positions of carbon 9″ and carbon 10″, that is, two adjacent R₃ may form an aromatic ring with carbon 9″ and carbon 10″, as shown in (F-33). Alternatively, the positions at which two adjacent R₃ are bonded to form a ring may be positions of carbon 1″ and carbon 2″, that is, two adjacent R₃ may form an aromatic ring with carbon 1″ and carbon 2″, as shown in (F-34). Alternatively, the positions at which two adjacent R₃ are bonded to form a ring may be positions of carbon 3″ and carbon 4″, that is, two adjacent R₃ may form an aromatic ring with carbon 3″ and carbon 4″, as shown in (F-35). It will be noted that the positions at which two adjacent R₃ are bonded to form a ring are represented by the carbon numbers. The numbers are only used for illustrating a relative relationship between the positions at which two adjacent R₃ are bonded to form a ring and the connection position between L₃ and the phenanthryl group, and are not limitations on the functional layer material F.

It will be noted that the structural formulas listed above are examples of the structure of the functional layer material F, but not limitations on the functional layer material F.

In some embodiments, in a case where q is greater than 2, two adjacent R₁ are capable of being bonded to form a ring.

It can be understood that in a case where two adjacent R₁ are bonded to form a ring, in the IB-1 part in the structure represented by the general formula (I), at least one benzene ring is fused with a ring bonded by two adjacent R₁. It will be noted that the position of the shared carbon atom when the benzene ring is fused with a ring bonded by two adjacent R₁ is not limited here.

The two adjacent R₁ are set to be capable of being bonded to form a ring. In an aspect, a portion connected to form a ring may increase an overall rigidity of the molecule of the functional layer material F, thereby having a rather high glass transition temperature Tg, increasing the stability of the material, and thus prolonging the life. In a second aspect, compared to a structure in which two adjacent R₁ exist but are not connected to form a ring, two adjacent R₁ being bonded to form a ring may appropriately reduce the three-dimensionality of the structure of the functional layer material F, thereby improving the mobility. In a third aspect, the setting of two adjacent R₁ being bonded to form a ring may increase richness of types of the functional layer material F, so that the functional layer material F may be combined with different types of electron transport materials to achieve carrier balance.

In some embodiments, in a case where n is greater than 2, two adjacent R₂ are capable of being bonded to form a ring.

It can be understood that in a case where two adjacent R₂ are bonded to form a ring, in the IC-1 part in the structure represented by the general formula (I), at least one benzene ring is fused with a ring bonded by two adjacent R₂. It will be noted that the position of the shared carbon atom when the benzene ring is fused with a ring bonded by two adjacent R₂ is not limited here.

The two adjacent R₂ are set to be capable of being bonded to form a ring. In an aspect, a portion connected to form a ring may increase an overall rigidity of the molecule of the functional layer material F, thereby having a rather high glass transition temperature Tg, increasing the stability of the material, and thus prolonging the life. In a second aspect, compared to a structure in which two adjacent R₂ exist but are not connected to form a ring, two adjacent R₂ being bonded to form a ring may appropriately reduce the three-dimensionality of the structure of the functional layer material F, thereby improving the mobility. In a third aspect, the setting of two adjacent R₂ being bonded to form a ring may increase richness of types of the functional layer material F, so that the functional layer material F may be combined with different types of electron transport materials to achieve carrier balance.

A synthesis process of the functional layer material F is described below in embodiments of the functional layer material F shown as the structural formulas (F-1) and (F-2).

In the related art, a carbon-carbon coupling reaction and a carbon-nitrogen coupling reaction are widely used in synthesis of organic materials. The carbon-carbon coupling reaction is, for example, a Suzuki coupling reaction, a Negishi coupling reaction, a Yamamoto coupling reaction, a Grignard coupling reaction, a Stille coupling reaction, or a Heck coupling reaction. The carbon-nitrogen coupling reaction is, for example, a Buchwald coupling reaction, an Ullmann coupling, a silylation reaction, a phosphating reaction, a borylation reaction, or a polycondensation reaction.

The above reaction is, for example, represented by a following general reaction formula (A):

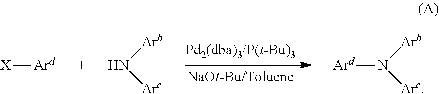

It will be noted that catalysts of the general reaction formula (A) are Pd₂(dba)₃ (tris(dibenzylideneacetone)dipalladium), P(t-Bu)₃ (tri-tert-butylphosphine) and NaOt-Bu (sodium tert-butoxide), and a reaction solvent is toluene. X is one of chlorine, bromine and iodine. Ar^{d}, Ar^{b} and Ar^{c} are groups that need to be connected through a coupling reaction.

For example, based on the above general reaction formula (A), a method for preparing a functional layer material (F-1) includes steps S1 to S3.

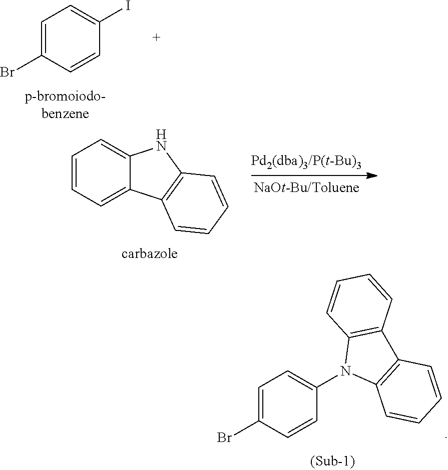 [SMILES: Brc1ccc(I)cc1 ; c1ccc2c(c1)[nH]c1ccccc12 ; Brc1ccc(-n2c3ccccc3c3ccccc32)cc1]

In S1 p-bromoiodobenzene (15 mmol), carbazole (15 mmol) and toluene (50 ml) are added into a reaction vessel, so that p-bromoiodobenzene and carbazole are dissolved in toluene. Then, tris(dibenzylideneacetone)dipalladium (Pd₂(dba)₃) (0.15 mmol), tri-tert-butylphosphine (P(t-Bu)₃) (0.8 mmol) and sodium tert-butoxide (t-BuONa) (45 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain an intermediate compound Sub-1 with a yield of 80%.

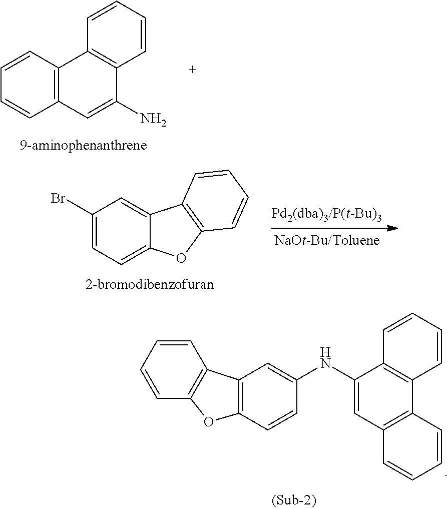 [SMILES: Nc1cc2ccccc2c2ccccc12 ; Brc1ccc2oc3ccccc3c2c1 ; c1ccc2c(c1)cc(Nc1ccc3oc4ccccc4c3c1)c1ccccc12]

In S2, 9-aminophenanthrene (15 mmol), 2-bromodibenzofuran (15 mmol) and toluene (50 ml) are added into the reaction vessel, so that 9-aminophenanthrene and 2-bromodibenzofuran are dissolved in toluene. Then, tris(dibenzylideneacetone)dipalladium (Pd₂(dba)₃) (0.15 mmol), tri-tert-butylphosphine (P(t-Bu)₃) (0.8 mmol) and sodium tert-butoxide (t-BuONa) (45 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain an intermediate compound Sub-2 with a yield of 78%.

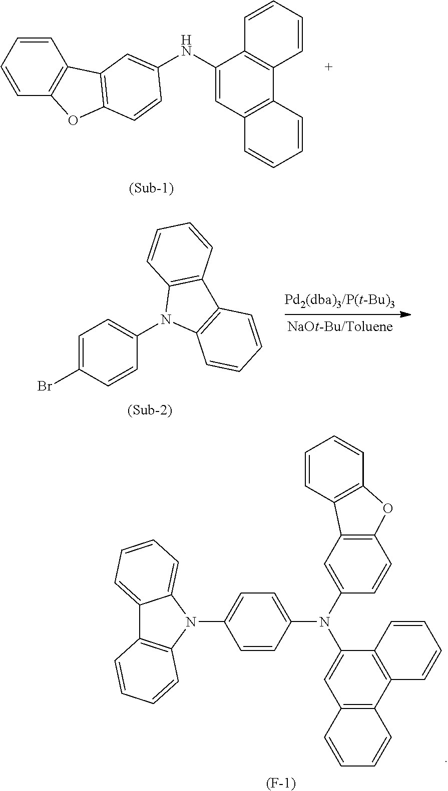 [SMILES: c1ccc2c(c1)cc(Nc1ccc3oc4ccccc4c3c1)c1ccccc12 ; Brc1ccc(-n2c3ccccc3c3ccccc32)cc1 ; c1ccc2c(c1)cc(N(c1ccc(-n3c4ccccc4c4ccccc43)cc1)c1ccc3oc4ccccc4c3c1)c1ccccc12 ; "(F-1)"]

In S3: an intermediate compound Sub-1 (12 mmol), an intermediate compound Sub-2 (12 mmol) and toluene (40 ml) are added into the reaction vessel, so that the intermediate compound Sub-1 and the intermediate compound Sub-2 are dissolved in toluene. Then, tris(dibenzylideneacetone)dipalladium (Pd₂(dba)₃) (0.12 mmol), tri-tert-butylphosphine (P(t-Bu)₃) (0.64 mmol) and sodium tert-butoxide (t-BuONa) (36 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain the functional layer material (F-1) with a yield of 75%.

For example, based on the above general reaction formula (A), a method for preparing a functional layer material (F-2) includes steps P1 to P3.

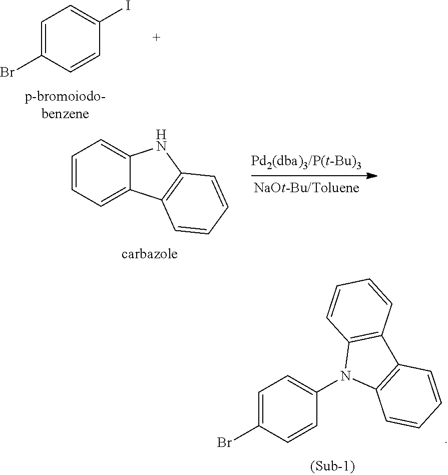 [SMILES: Brc1ccc(I)cc1 ; c1ccc2c(c1)[nH]c1ccccc12 ; Brc1ccc(-n2c3ccccc3c3ccccc32)cc1]

In P1: p-bromoiodobenzene (15 mmol), carbazole (15 mmol) and toluene (50 ml) are added into a reaction vessel, so that p-bromoiodobenzene and carbazole are dissolved in toluene. Then, tris(dibenzylideneacetone)dipalladium (Pd₂(dba)₃) (0.15 mmol), tri-tert-butylphosphine (P(t-Bu)₃) (0.8 mmol) and sodium tert-butoxide (t-BuONa) (45 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain an intermediate compound Sub-1 with a yield of 80%.

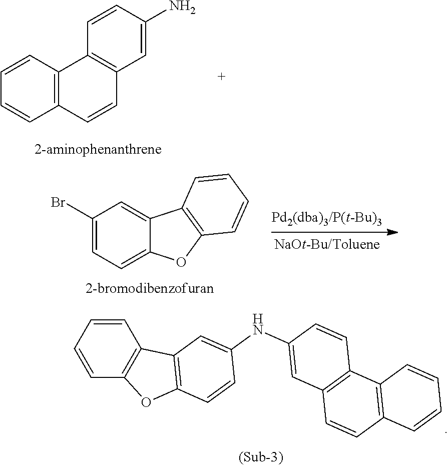 [SMILES: Nc1ccc2c(ccc3ccccc32)c1 ; Brc1ccc2oc3ccccc3c2c1 ; c1ccc2c(c1)ccc1cc(Nc3ccc4oc5ccccc5c4c3)ccc12]

In P2, 2-aminophenanthrene (15 mmol), 2-bromodibenzofuran (15 mmol) and toluene (50 ml) are added into the reaction vessel, so that 2-aminophenanthrene and 2-bromodibenzofuran are dissolved in toluene. Then, tris(dibenzylideneacetone)dipalladium (Pd₂(dba)₃) (0.15 mmol), tri-tert-butylphosphine (P(t-Bu)₃) (0.8 mmol) and sodium tert-butoxide (t-BuONa) (45 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain an intermediate compound Sub-3 with a yield of 75%.

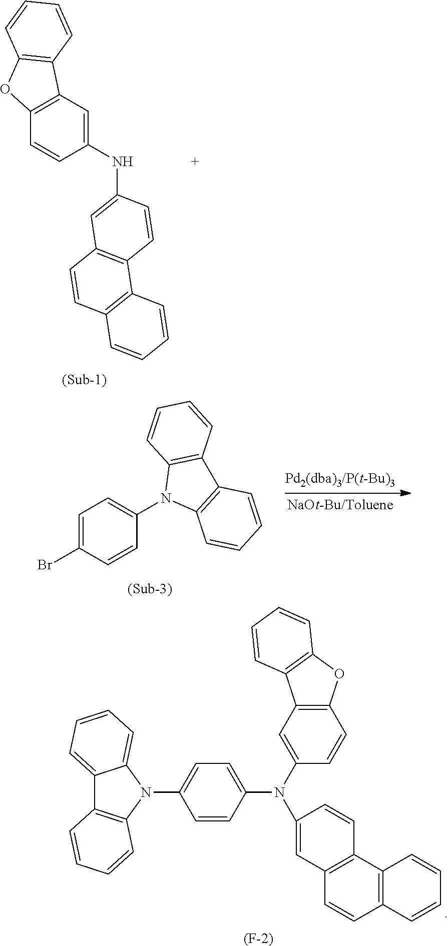 [SMILES: c1ccc2c(c1)ccc1cc(Nc3ccc4oc5ccccc5c4c3)ccc12 ; Brc1ccc(-n2c3ccccc3c3ccccc32)cc1 ; c1ccc2c(c1)ccc1cc(N(c3ccc(-n4c5ccccc5c5ccccc54)cc3)c3ccc4oc5ccccc5c4c3)ccc12 ; "(F-2)"]

In P3, an intermediate compound Sub-1 (12 mmol), an intermediate compound Sub-3 (12 mmol) and toluene (40 ml) are added into the reaction vessel, so that the intermediate compound Sub-1 and the intermediate compound Sub-3 are dissolved in toluene. Then, tris(dibenzylideneacetone)dipalladium (Pd₂(dba)₃) (0.12 mmol), tri-tert-butylphosphine (P(t-Bu)₃) (0.64 mmol) and sodium tert-butoxide (t-BuONa) (36 mmol) are added under a nitrogen atmosphere. After addition, a reaction temperature is slowly raised to 110° C., and the mixture is stirred for 10 h at this temperature. Then, distilled water is added to a reaction solution, and the reaction solution is extracted with ethyl acetate. Next, the extracted organic layer is dried using magnesium sulfate, and the solvent is removed using a rotary evaporator. Then, the remaining materials are purified using column chromatography to obtain the functional layer material (F-2) with a yield of 77%.

In another aspect, as shown in FIG. 1, a light-emitting device 100 is provided. The structure of the light-emitting device 100 is described below.

Some embodiments of the present disclosure provide a light-emitting device 100. As shown in FIG. 1, the light-emitting device 100 includes a cathode 110 and an anode 120 that are provided oppositely, and at least one light-emitting unit 130 provided between the cathode 110 and the anode 120. The light-emitting unit 130 includes a light-emitting layer 131 and a first type of functional layer 132 disposed on a side of the light-emitting layer 131 proximate to the anode 120. A material of the first type of functional layer 132 includes the functional layer material F as described in any of the above embodiments. The light-emitting device 100 is, for example, an OLED light-emitting device.

The light-emitting device 100 includes at least one light-emitting unit 130. In a case where the light-emitting device 100 includes a single light-emitting unit 130, the light-emitting device 100 is a single-layer light-emitting device 100. In a case where the light-emitting device 100 includes a plurality of light-emitting units 130, the light-emitting device 100 is a stacked light-emitting device 100, and the plurality of light-emitting units 130 of the stacked light-emitting device 100 are sequentially connected in a direction perpendicular to a light-emitting surface, where the direction perpendicular to the light-emitting surface is a first direction P as shown in FIG. 1.

Based on the above structure, a light-emitting principle of the light-emitting device 100 is as follows: through a circuit connected by the cathode 110 and the anode 120, holes are injected into the light-emitting layer 131 using the anode 120, and electrons are injected into the light-emitting layer 131 using the cathode 110; the injected electrons and holes form excitons (i.e., electron-hole pairs) in the light-emitting layer 131, and the excitons transition back to a ground state by radiation to emit photons. It can be seen that during light emission of the light-emitting device 100, effective charge injection and rapid charge transport are both indispensable. The above-mentioned charges are holes or electrons.

The first type of functional layer 132 is provided on a side of the light-emitting layer 131 proximate to the anode 120, and is configured to transport holes and/or block electrons. In this way, effective hole injection and rapid hole transport may be achieved; moreover, the electrons may be allowed to stay in the light-emitting layer 131 as much as possible, so that electrons and holes are balanced during light emission, thereby improving the luminous efficiency of the light-emitting layer 131.

The material of the first type of functional layer 132 is provided to include the functional layer material F. In a first aspect, the functional layer material F may play a role of transporting holes and/or blocking electrons, thereby achieving effective hole injection and rapid hole transport. In a second aspect, since the structural formula of the functional layer material F includes the second substitution unit IB, the heteroatom X or Y in the second substitution unit IB may improve the hole transport ability of the functional layer material F, increase the amount of holes transported to the light-emitting layer 131, improve the recombination probability of holes and electrons in the light-emitting layer 131, thereby improving the emission efficiency. Moreover, since the second substitution unit IB has a relatively high triplet energy level T1, the functional layer material F may have a relatively high triplet energy level T1, so as to block excitons in the light-emitting layer 131 from transporting towards a direction close to the first type of functional layer 132, so that more excitons may stay in the light-emitting layer 131, thereby effectively improving the luminous efficiency of the light-emitting device 100. In a third aspect, since the structural formula of the functional layer material F includes the third substitution unit IC containing a carbazolyl group, the functional layer material F may have a suitable highest occupied molecular orbital (HOMO) energy level, thereby reducing an energy gap (GAP) that needs to be overcome during transport of holes from the first type of functional layer 132 to the light-emitting layer 131. In this way, a voltage for driving the light-emitting device 100 to emit light may be effectively reduced, and thus the power consumption of the light-emitting device 100 may be reduced. In a fourth aspect, since the structural formula of the functional layer material F includes the first substitution unit IA containing a fused ring aryl group, the functional layer material F has relatively good conjugation. Due to a synergistic effect of the second substitution unit IB and the third substitution unit IC, the light-emitting device 100 may have a relatively high luminous efficiency, a relatively low voltage and a long service life.

It will be noted that in a case where the light-emitting device 100 is a stacked light-emitting device, the first type of functional layer 132 including the functional layer material F may be located in a light-emitting unit 130 of the plurality of light-emitting units 130, or located in some light-emitting units 130 of the plurality of light-emitting units 130, or located in each light-emitting unit 130 of the plurality of light-emitting units 130. That is, in a case where the light-emitting device 100 is a stacked light-emitting device 100, the number of the light-emitting units 130 including the functional layer material F is not limited.

In some embodiments, as shown in FIG. 1, the first type of functional layer 132 includes a plurality of functional sub-layers 1321, and at least one functional sub-layer 1321 of the plurality of functional sub-layers 1321 includes the functional layer material F.

It can be understood that in a case where the first type of functional layer 132 includes a plurality of functional sub-layers 1321, the at least one functional sub-layer 1321 of the plurality of functional sub-layers 1321 is provided to include the functional layer material F, and thus the luminous efficiency of the light-emitting device 100 may be effectively improved, the voltage for driving the light-emitting device 100 to emit light may be effectively reduced, and the service life of the light-emitting device 100 may be prolonged.

For example, as shown in FIG. 1, the first type of functional layer 132 includes any one or more of a hole injection functional layer 13211, a hole transport functional layer 13212 and an electron blocking functional layer 13213.

In some embodiments, as shown in FIG. 1, the plurality of functional sub-layers 1321 include an electron blocking functional layer 13213, a hole injection functional layer 13211 and a hole transport functional layer 13212 that are stacked. The hole injection functional layer 13211, the hole transport functional layer 13212 and the electron blocking functional layer 13213 are arranged in sequence in a direction away from the anode 120. In the hole injection functional layer 13211, the hole transport functional layer 13212 and the electron blocking functional layer 13213, at least the electron blocking functional layer 13213 includes the functional layer material F.

Based on the structure shown in the above general formulas, the functional layer material F has a good exciton blocking property. In a case where at least the electron blocking functional layer 13213 in the plurality of functional sub-layers 1321 of the first type of functional layer 132 includes the functional layer material F, the functional layer material F may be located proximate to the light-emitting layer 131 to play a function of blocking excitons. In this way, the excitons may be allowed to stay in the light-emitting layer 131 as much as possible, thereby further improving the luminous efficiency of the light-emitting layer 131.

In some embodiments, as shown in FIG. 1, the light-emitting unit 130 further includes a second type of functional layer 133. The second type of functional layer 133 is located on a side of the light-emitting layer 131 proximate to the cathode 110 and is configured to transport electrons or block holes. In this way, effective electron injection and rapid electron transport may be achieved; moreover, the holes may be blocked, which is beneficial to balance between electrons and holes during light emission.

For example, the second type of functional layer 133 includes any one or more of an electron injection functional layer 1331, an electron transport functional layer 1332 and a hole blocking functional layer 1333.

As shown in FIG. 1, in a case where the second type of functional layer 133 includes an electron injection functional layer 1331, an electron transport functional layer 1332 and a hole blocking functional layer 1333, the second type of functional layer 133 is arranged in a way of that, for example, the electron injection functional layer 1331, the electron transport functional layer 1332 and the hole blocking functional layer 133 are arranged in sequence in a direction away from the cathode 110.

In some embodiments, the light-emitting layer 131 is configured to emit blue light, and a material of the light-emitting layer 131 includes a host material and a guest material. The host material H has a structural formula as follows:

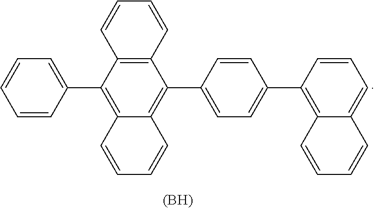

The guest material BD has a structural formula as follows:

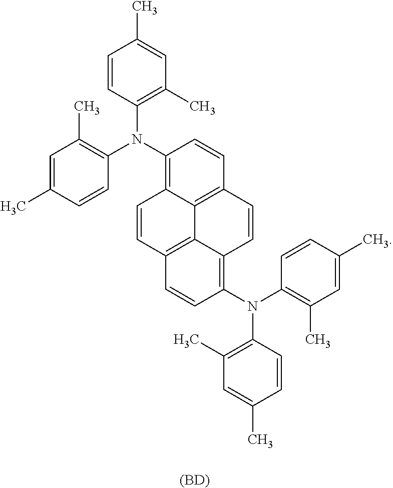 [SMILES: Cc1ccc(N(c2ccc(C)cc2C)c2ccc3ccc4c(N(c5ccc(C)cc5C)c5ccc(C)cc5C)ccc5ccc2c3c54)c(C)c1 ; "(BD)"]

The material of the light-emitting layer 131 includes the host material and the guest material. For example, the host material is configured to transmit energy through fluorescence resonance energy transfer, and the guest material is configured to obtain the energy transmitted by the host material through fluorescence resonance energy transfer to emit blue light.

It can be understood that in a case where the light-emitting layer 131 is configured to emit blue light, the light-emitting device 100 is a blue light-emitting device. In a case where the light-emitting device 100 is the blue light-emitting device, the provision of the material of the first type of functional layer 132 including the functional layer material F may achieve following effects: since the light-emitting layer 131 of the blue light-emitting device contains a relatively large amount of electrons, the provision of the material of the first type of functional layer 132 including the functional layer material F may increase the amount of holes transported to the light-emitting layer 131, increase a recombination probability of holes and electrons in the light-emitting layer 131, and improve the emission efficiency; moreover, since the material of the light-emitting layer 131 of the blue light-emitting device has a relatively deep highest occupied molecular orbital (HOMO) energy level, and the functional layer material F containing a carbazolyl group also has a relatively deep highest occupied molecular orbital (HOMO) energy level, an energy gap (GAP) that needs to be overcome during transport of holes from the first type of functional layer 132 to the light-emitting layer 131 may be reduced, so that the voltage for driving the light-emitting device 100 to emit light may be effectively reduced, and thus the power consumption of the light-emitting device 100 may be reduced. It will be noted that the relatively deep highest occupied molecular orbital (HOMO) energy level mentioned here means that a relatively large absolute value of the highest occupied molecular orbital (HOMO) energy level.

In order to objectively evaluate technical effects of the embodiments of the present disclosure, technical solutions provided in the present disclosure will be exemplarily described in detail below through following Experimental examples and Comparative example.

The materials involved in the following Experimental examples and Comparative example include:

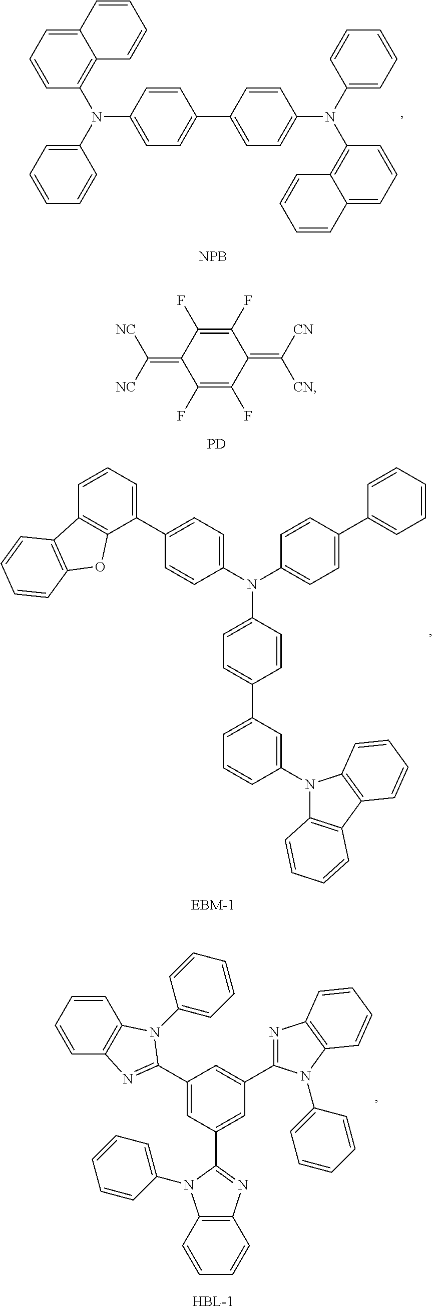

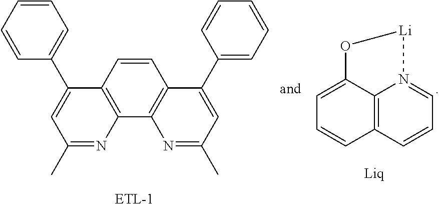

It will be noted that NPB, PD, EBM-1, HBL-1, ETL-1 and Liq in the above structural formulas is an antonomasia of each structural formula, and is not a part of structure of the structural formula.

The following Experimental examples and Comparative example calculate and compare the highest occupied molecular orbital (HOMO) energy levels and triplet energy levels (T1) of the functional layer materials F.

In the following Experimental examples and Comparative example, the highest occupied molecular orbital (HOMO) energy levels are calculated using the same method, the triplet energy levels (T1) are calculated using the same method, and the calculation results are shown in Table 1 below.

TABLE 1

| | HOMO | T1 |
|---|---|---|
| F-1 | −5.00 | 2.64 |
| F-3 | −4.98 | 2.66 |
| F-13 | −4.99 | 2.63 |
| F-48 | −4.91 | 2.72 |
| F-27 | −4.97 | 2.62 |
| EBM-1 | −4.96 | 2.60 |

It will be noted that units of the highest occupied molecular orbital (HOMO) energy level and triplet energy level (T1) in Table 1 is electron volt with a symbol of eV. As for structural formulas represented by F-x (x is a positive integer) and EBM-1, reference is made to the above contents, and details are not repeated here.

It can be seen from Table 1 that the highest occupied molecular orbital (HOMO) energy levels of the five functional layer materials F are in a range of −4.91 eV to −5.00 eV, inclusive, and the five functional layer materials F all have relatively deep highest occupied molecular orbital (HOMO) energy levels. In this way, the energy gap (GAP) that needs to be overcome during transport of holes from the first type of functional layer 132 to the light-emitting layer 131 may be reduced, the voltage for driving the light-emitting device 100 to emit light may be effectively reduced, and thus the power consumption of the light-emitting device 100 may be effectively reduced.

It can be seen from Table 1 that the triplet energy levels (T1) of the five functional layer materials F are in a range of 2.62 eV to 2.72 eV, inclusive, and are all greater than a triplet energy level of (EBM-1), which indicate that the functional layer materials F have relatively high triplet energy levels T1. In this way, excitons in the light-emitting layer 131 may be blocked from transporting towards a direction close to the first type of functional layer 132, so that more excitons may stay in the light-emitting layer 131, and the luminous efficiency of the light-emitting device 100 may be effectively improved.

The following Experimental examples and Comparative example use different materials to make the electron blocking functional layers 13213 of the light-emitting devices 100, and compare voltages, luminous efficiencies and device lives of the light-emitting devices 100.

In the following Comparative example and Experimental examples, the light-emitting devices 100 have the same test conditions, and the same type of materials have the same test conditions.

As shown in FIG. 1, the light-emitting devices 100 in the following Comparative example and Experimental examples each include an anode 120 and a cathode 110 that are provided oppositely, and a light-emitting unit 130 provided between the cathode 110 and the anode 120. The light-emitting unit 130 includes a first type of functional layer 132, a light-emitting layer 131 and a second type of functional layer 133. The first type of functional layer 132 includes a hole injection functional layer 13211, a hole transport functional layer 13212 and an electron blocking functional layer 13213 that are sequentially arranged in a direction from the anode 120 to the cathode 110. The second type of functional layer 133 includes an electron injection functional layer 1331, an electron transport functional layer 1332 and a hole blocking functional layer 1333 that are sequentially arranged in a direction from the cathode 110 to the anode 120.

In the following Comparative example and Experimental examples, thicknesses of the hole injection functional layer 13211, the hole transport functional layer 13212, the electron blocking functional layer 13213, the light-emitting layer 131, the hole blocking functional layer 1333, the electron transport functional layer 1332 and the electron injection functional layer 1331 that are stacked in a direction from the anode 120 to the cathode 110 are 10 nm, 80 nm, 10 nm, 20 nm, 5 nm, 30 nm and 1 nm, respectively.

The portions, with the same materials, of film layers in the Comparative example and Experimental examples will be described below. In the following Comparative example and Experimental examples, the materials of the anodes 120 are each indium tin oxide (ITO). The materials of the hole injection functional layers 13211 each include a material represented by the structural formula (NPB) and a material represented by the structural formula (PD), and a mass ratio of the material represented by the structural formula (NPB) to the material represented by the structural formula (PD) is 97:3. The materials of the hole transport functional layers 13212 are each a material represented by the structural formula (NPB). The materials of the light-emitting layers 131 each include a host material and a guest material, the structure of the host material is shown as the above structural formula (BH), the structure of the guest material is shown as the above structural formula (BD), and a mass ratio of the host material to the guest material is 99:1. The structures of the materials of the hole blocking functional layers 1333 are each shown as the structural formula (HBL-1), and the material is 1,3,5-Tris(1-phenyl-1H-benzimidazol-2-yl)benzene (TPBI). The materials of the electron transport functional layers 1332 each include a material represented by the structural formula (ETL-1) and a material represented by the structural formula (Liq), and a mass ratio of the material represented by the structural formula (ETL-1) to the material represented by the structural formula (Liq) is 1:1. The materials of the electron injection functional layers 1331 are each lithium fluoride (LiF). The materials of the cathodes 110 are each a magnesium-silver alloy.

In the following Comparative example and Experimental examples, the materials of the electron blocking functional layers 13213 are set separately. Specifically, the structure of the material of the electron blocking functional layer 13213 in Comparative Example 1 is shown as the structural formula (EBM-1), and the structures of the materials of the electron blocking functional layers 13213 in Experimental examples 1 to 5 are shown as the structural formulas (F-1), (F-3), (F-48), (F-45) and (F-67), respectively.

In order to more clearly describe a difference between the film layer material structures used in the light-emitting devices 100 in the Experimental examples and Comparative example, the film layer material structures used in the light-emitting devices 100 in the Experimental examples and Comparative example are shown more clearly in Table 2 below.

TABLE 2

| Device structure | HI | HT | EBL | EML | HB | ET | EI |
|---|---|---|---|---|---|---|---|
| Comparative | NPB/PD | NPB | EBM-1 | BH/BD | HBL-1 | ETL-1/Liq | LiF |
| example 1 | | | | | | | |
| Experimental | | | F-1 | | | | |
| example 1 | | | | | | | |
| Experimental | | | F-3 | | | | |
| example 2 | | | | | | | |
| Experimental | | | F-48 | | | | |
| example 3 | | | | | | | |
| Experimental | | | F-45 | | | | |
| example 4 | | | | | | | |
| Experimental | | | F-67 | | | | |
| example 5 | | | | | | | |

It will be noted that in Table 2, HI represents the hole injection functional layer 13211, HT represents the hole transport functional layer 13212, EBL represents the electron blocking functional layer 13213, EML represents the light-emitting layer 131, HB represents the hole blocking functional layer 1333, ET represents the electron transport functional layer 1332, and EI represents the electron injection functional layer 1331. “A/B” in Table 2 means that the material of the film layer includes a material A and a material B. For example, the material of HI is NPB/PD, which means that the material of the hole injection functional layer 13211 includes a material with the structural formula of (NPB) and a material with the structural formula of (PD). “A-x” in Table 2 means that a corresponding structural formula is A-x. For example, a content in a sub-grid corresponding to the EBL in Experimental example 1 is “F-1”, which means that the material of the electron blocking functional layer 13213 in Experimental example 1 is a functional layer material F with the structural formula shown in (F-1). As for structural formulas represented by F-x (x is a positive integer), NPB, PD, EBM-1, BH, BD, HBL-1, ETL-1 and Liq, reference is made to the above contents, and details are not repeated here.

Based on the above materials, voltages (V), luminous efficiencies (Eff.) and device lives (LT) of the light-emitting devices 100 in Experimental examples 1 to 5 and Comparative example 1 are tested. The data results of voltages (V), luminous efficiencies (Eff.) and device lives (LT) are based on comparative example 1 as a reference, and the test results are shown in Table 3 below.

TABLE 3

| Device structure | V | Eff. | LT |
|---|---|---|---|
| Comparative example 1 | 100% | 100% | 100% |
| Experimental example 1 | 95% | 132% | 135% |
| Experimental example 2 | 94% | 129% | 143% |
| Experimental example 3 | 96% | 128% | 142% |
| Experimental example 4 | 95% | 129% | 168% |
| Experimental example 5 | 96% | 135% | 138% |

It can be seen from Table 3 that taking the test data of Comparative Example 1 as a reference, voltages of Experimental examples 1 to 5 are in a range of 94% to 96%, inclusive, which is lower than a voltage of Comparative Example 1. It can be seen that the voltages of Experimental examples 1 to 5 are relatively low, which may reduce the power consumptions of the light-emitting devices 100. Luminous efficiencies of Experimental examples 1 to 5 are in a range of 128% to 135%, inclusive, which is greater than a luminous efficiency of Comparative Example 1. It can be seen that the luminous efficiencies of Experimental examples 1 to 5 are significantly improved. Device lives of Experimental examples 1 to 5 are in a range of 135% to 168%, inclusive, which is greater than a device life of Comparative Example 1. It can be seen that the device lives of Experimental examples 1 to 5 are significantly improved.

Here, a device life of Experimental example 4 is 168%, and is significantly higher than that of other Experimental examples and Comparative example. This is because the structure of the material of the electron blocking functional layer 13213 of Experimental example 4 is as shown in the structural formula (F-45). The second substitution unit IB contains a deuterium substituent, and therefore molecular vibration may be effectively suppressed, a bond length may be reduced, a bond energy may be enhanced, so that the molecular stability may be improved. In this way, the stability of the functional layer material F may be improved, and the service life of the light-emitting device 100 may further be prolonged.

Therefore, it can be seen from the above tests that in the light-emitting device 100 provided by the present disclosure, an electron blocking functional layer 13213 containing the functional layer material F is provided, and the second substitution unit IB of the functional layer material F contains a dibenzofuran structure or a dibenzothiophene structure. In this way, the heteroatom oxygen in the dibenzofuran structure or the heteroatom sulfur in the dibenzothiophene structure may improve a hole transport ability of the functional layer material F, increase the amount of holes transported to the light-emitting layer 131, improve a recombination probability of holes and electrons in the light-emitting layer 131, thereby improving an emission efficiency. Moreover, the second substitution unit IB is provided to be connected to nitrogen, so that the functional layer material F has a relatively high triplet energy level T1, so as to block excitons in the light-emitting layer 131 from transporting towards a direction close to the first type of functional layer 132, and thus more excitons may stay in the light-emitting layer 131, thereby effectively improving the luminous efficiency of the light-emitting device 100. In addition, the third substituent unit IC of the functional layer material F contains a carbazolyl group connected to nitrogen, so that the functional layer material F have a relatively deep highest occupied molecular orbital (HOMO) energy level, so as to reduce an energy gap (GAP) that needs to be overcome during transport of holes from the first type of functional layer 132 to the light-emitting layer 131, which is beneficial to reducing the voltage of the light-emitting device 100, thereby reducing the power consumption of the light-emitting device 100.

It can be seen that in the light-emitting device 100 provided in the present disclosure, the provision of the first type of functional layer 132 containing the functional layer material F may effectively improve the device life and the luminous efficiency, thereby effectively reducing the voltage and the power consumption of the light-emitting device 100.

Some embodiments of the present disclosure further provide a display panel 1000. As shown in FIG. 2, the display panel 1000 includes the light-emitting device 100 as described in any of the above embodiments.

In some embodiments, as shown in FIG. 2, the display panel 1000 includes a base substrate 200, and an array layer 300, a light-emitting functional layer 400 and an encapsulation layer 500 that are provided on the base substrate 200. The light-emitting functional layer 400 is located on a side of the array layer 300. The array layer 300 includes a plurality of pixel driving circuits 310, and the pixel driving circuit 310 includes a plurality of transistors TFT. The light-emitting functional layer 400 includes a plurality of light-emitting devices 100, and the plurality of light-emitting devices 100 are arranged in a second direction Q.

In some examples, as shown in FIG. 2, the plurality of pixel driving circuits 310 and the plurality of light-emitting devices 100 may be coupled in one-to-one correspondence. In some other examples, a pixel driving circuit 310 may be coupled to multiple light-emitting devices 100, alternatively, multiple pixel driving circuits 310 may be coupled to a light-emitting device 100.

For example, in the display panel 1000, the pixel driving circuit 310 may generate a driving current. Each light-emitting device 100 may emit light due to an driving action of the driving current generated by the corresponding pixel driving circuit 310, and lights emitted by the plurality of light-emitting devices 100 cooperate to make the display panel 1000 achieve the display function.

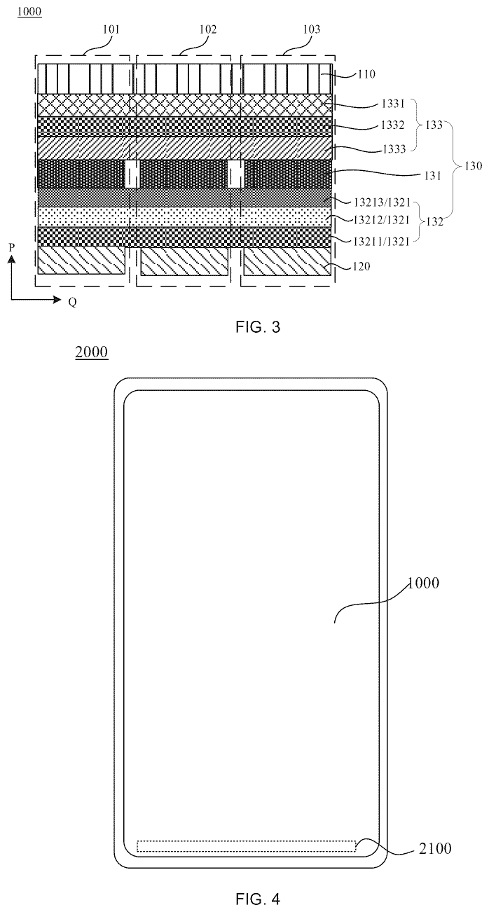

In some embodiments, as shown in FIG. 3, the plurality of light-emitting devices 100 of the display panel 1000 include a red light-emitting device 101, a green light-emitting device 102 and a blue light-emitting device 103. Due to action of the driving voltage, the red light-emitting device 101 emits red light, the green light-emitting device 102 emits green light, and the blue light-emitting device 103 emits blue light.

It will be noted that FIG. 3 is a simplified schematic diagram obtained after other film layers in the display panel 1000 except the film layers related to the light-emitting device 100 are removed. In FIG. 3, the plurality of light-emitting devices 100 are, for example, a red light-emitting device 101, a green light-emitting device 102 and a blue light-emitting device 103. Each light-emitting device 100 includes a respective light-emitting layer 131 and a respective anode 120. In addition to the light-emitting layer 131 and the anode 120, other film layers are connected to form a whole for sharing.

The beneficial effects of the display panel 1000 are the same as the beneficial effects of the light-emitting device 100 provided in the foregoing embodiments of the present disclosure, and details are not described here again.

As shown in FIG. 4, some embodiments of the present disclosure further provide a display apparatus 2000, and the display apparatus 2000 includes the display panel 1000.

In some examples, the display apparatus 2000 may be an organic light-emitting diode (OLED) display apparatus.

For example, as shown in FIG. 4, the display apparatus 2000 further includes a driver chip 2100. The driver chip 2100 is used to drive the display panel 1000 to display.

In some examples, the driver chip 2100 is electrically connected to the display panel 1000 through a flexible circuit board, and is bent to a back of the display panel 1000 along with the flexible circuit board to narrow a border of the display apparatus 2000 and increase an area of a display area. The dashed line in FIG. 4 illustrates that the driver chip 2100 is located on the back of the display panel 1000.

In addition, the display apparatus 2000 may further include an under-screen camera, an under-screen fingerprint recognition sensor, and the like, so that the display apparatus 2000 is able to implement various functions such as taking pictures, video recording, fingerprint recognition or face recognition.

The display apparatus 2000 may be any display apparatus that displays images whether in motion (such as a video) or fixed (such as a still image), and regardless of text or image. More specifically, it is expected that the display apparatus 2000 in the embodiments may be implemented in or associated with a variety of electronic devices. The variety of electronic devices may include (but are not limit to), for example, mobile phones, wireless devices, personal digital assistants (PDAs), hand-held or portable computers, global positioning system (GPS) receivers/navigators, cameras, MPEG-4 Part 14 (MP4) video players, video cameras, game consoles, watches, clocks, calculators, TV monitors, flat-panel displays, computer monitors, car displays (e.g., odometer displays), navigators, cockpit controllers and/or displays, camera view displays (e.g., display of rear view camera in vehicles), electronic photos, electronic billboards or signs, projectors, architectural structures, packaging and aesthetic structures (e.g., displays for displaying an image of a piece of jewelry), etc.

The foregoing descriptions are merely specific implementations of the present disclosure, but the protection scope of the present disclosure is not limited thereto. Changes or replacements that any person skilled in the art could conceive of within the technical scope of the present disclosure shall be included in the protection scope of the present disclosure. Therefore, the protection scope of the present disclosure shall be subject to the protection scope of the claims.

1. A functional layer material, selected from any of structures represented by a general formula (I); 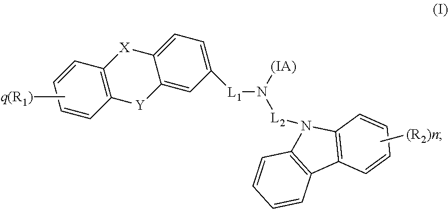
wherein IA represents a first substitution unit that includes at least one fused ring aryl group;
X is selected from any of O, S and Se;
Y is selected from any of O, S, N(R₄), C(R₅R₆) and a single bond, and X and Y are same or different;
L₁ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups;
L₂ is selected from any of substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; and L₂ and L₁ are same or different;
R₁, R₂, R₄, R₅ and R₆ are same or different, and are each independently selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups;
n takes a value of a positive integer greater than or equal to 1; and
q takes a value of a positive integer greater than or equal to 1.

2. The functional layer material according to claim 1, wherein the functional layer material is selected from any of structures represented by a general formula (II); 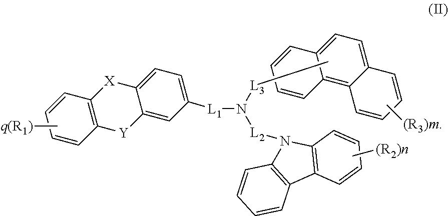
wherein, L₃ is selected from any of a single bond, substituted or unsubstituted C6 to C30 arylene groups, substituted or unsubstituted C1 to C30 heteroarylene groups, substituted or unsubstituted C7 to C30 aralkylene groups, and substituted or unsubstituted C2 to C30 heteroaralkylene groups; L₃ and L₁ are same or different, and L₃ and L₂ are same or different;
R₃ is selected from hydrogen, deuterium, substituted or unsubstituted C1 to C39 alkyl groups, substituted or unsubstituted C2 to C39 alkenyl groups, substituted or unsubstituted C2 to C39 alkynyl groups, substituted or unsubstituted C6 to C39 aryl groups, substituted or unsubstituted C5 to C60 heteroaryl groups, substituted or unsubstituted C6 to C60 aryloxy groups, substituted or unsubstituted C1 to C39 alkoxy groups, substituted or unsubstituted C6 to C39 arylamine groups, substituted or unsubstituted C3 to C39 cycloalkyl groups, substituted or unsubstituted C3 to C39 heterocyclylalkyl groups, substituted or unsubstituted C1 to C39 alkylsilyl groups, substituted or unsubstituted C1 to C39 alkyl boryl groups, substituted or unsubstituted C6 to C39 aryl boryl groups, substituted or unsubstituted C6 to C39 arylphosphino groups, and substituted or unsubstituted C6 to C39 arylsilyl groups; and
m takes a value of a positive integer greater than or equal to 1.

3. The functional layer material according to claim 1, wherein X is oxygen and Y is a single bond; or
X is sulfur and Y is a single bond.

4. The functional layer material according to claim 1, wherein X is oxygen and Y is oxygen; or
X is oxygen and Y is sulfur.

5. The functional layer material according to claim 2, wherein the functional layer material is selected from any of structures represented by a general formula (III);

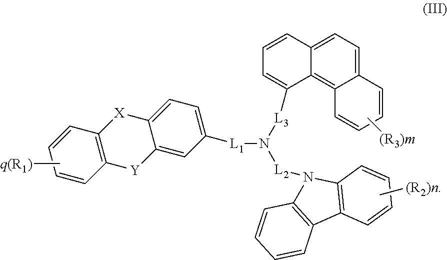

6. The functional layer material according to claim 2, wherein the functional layer material is selected from any of structures represented by a general formula (IV);

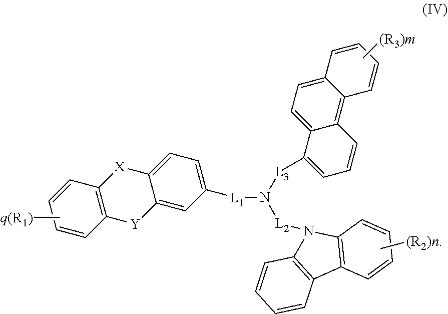

7. The functional layer material according to claim 1, wherein the structures represented by the general formula (I) contain at least one deuterium.

8. The functional layer material according to claim 2, wherein m is greater than, two adjacent R₃ are bonded to be a ring.

9. The functional layer material according to claim 1, wherein q is greater than 2, two adjacent R₁ are bonded to be a ring.

10. The functional layer material according to claim 1, wherein n is greater than 2, two adjacent R₂ are bonded to be a ring.

11. The functional layer material according to claim 1, wherein the functional layer material is used to transport holes and/or block electrons.

12. A light-emitting device, comprising: a cathode and an anode that are opposite, and at least one light-emitting unit disposed between the cathode and the anode, wherein
the light-emitting unit includes a light-emitting layer and a first type of functional layer disposed on a side of the light-emitting layer proximate to the anode; a material of the first type of functional layer includes the functional layer material according to claim 1.

13. The light-emitting device according to claim 12, wherein the first type of functional layer includes a plurality of functional sub-layers, and at least one functional sub-layer in the plurality of functional sub-layers includes the functional layer material.

14. The light-emitting device according to claim 13, wherein the plurality of functional sub-layers include an electron blocking functional layer, a hole injection functional layer and a hole transport functional layer that are stacked; the hole injection functional layer, the hole transport functional layer and the electron blocking functional layer are arranged in sequence in a direction away from the anode; and
in the hole injection functional layer, the hole transport functional layer and the electron blocking functional layer, at least the electron blocking functional layer includes the functional layer material.

15. The light-emitting device according to claim 12, wherein the light-emitting layer is configured to emit blue light, and a material of the light-emitting layer includes a host material and a guest material;
a structural formula of the host material is as follows: 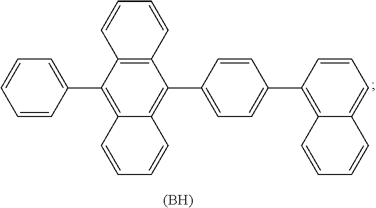
a structural formula of the guest material is as follows:

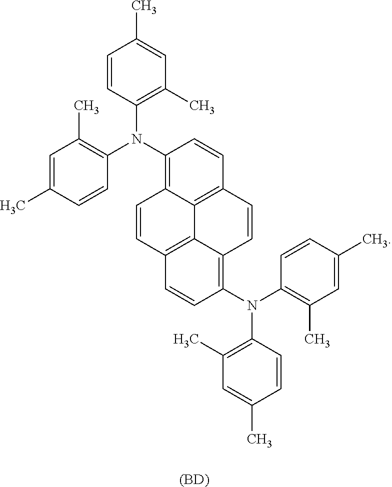 [SMILES: Cc1ccc(N(c2ccc(C)cc2C)c2ccc3ccc4c(N(c5ccc(C)cc5C)c5ccc(C)cc5C)ccc5ccc2c3c54)c(C)c1 ; "(BD)"]

16. A display panel, comprising a plurality of light-emitting devices each according to claim 12; and
pixel driving circuits electrically connected to the light-emitting devices, the pixel driving circuits being used to drive the light-emitting devices to emit light.

17. The functional layer material according to claim 2, wherein the structures represented by the general formula (II) contain at least one deuterium.

18. The functional layer material according to claim 5, wherein the structures represented by the general formula (III) contain at least one deuterium.

19. The functional layer material according to claim 6, wherein the structures represented by the general formula (IV) contain at least one deuterium.

20. A display apparatus, comprising:
the display panel according to claim 16; and
a driver chip electrically connect to the display panel and configured to drive the display panel to display.
